(19) United States
(12) Patent Application Publication
(10) Pub. No.: US 2026/0366058 A1
(43) Pub. Date: Nov. 19, 2026

(54) METHODS, SYSTEMS, AND COMPOSITIONS RELATING TO PREDICTING PROBIOTIC STRAINS FOR RESOLVING DISEASE

(71) Applicant: University of Virginia Patent Foundation, Charlottesville, VA (US)

(72) Inventors: Jason Aaron Papin, Charlottesville, VA (US); Emma Margaret Glass, Charlottesville, VA (US)

(21) Appl. No.: 19/676,484

(22) Filed: May 13, 2026

Related U.S. Application Data
(60) Provisional application No. 63/805,096, filed on May 13, 2025.

(51) Int. Cl. C12Q 1/04; G16B 20/00

(52) U.S. Cl. CPC C12Q 1/04; G16B 20/00

(57) ABSTRACT

Described herein are systems, methods, and computer readable medium for, among other things, predictive platforms for novel microbial probiotics. Such systems, methods, and computer readable medium can be utilized for identifying candidate probiotics and/or microbiota for administration against a candidate bacterial infection in particular. In embodiment, the bacterial infection is bacterial vaginosis. An aspect of the present disclosure also provides a system, method and computer readable medium for, among other things, algorithms/models that enabled the predictions of the specific combinations of bacteria and putative therapeutic targets to enable the therapeutic intervention.

### CROSS REFERENCE TO RELATED APPLICATION[S]

This application claims priority to, and the benefit of, U.S. Provisional Patent Application Ser. No. 63/805,096 filed on May 13, 2025, which is incorporated herein by reference in its entirety as if fully set forth herein.

### STATEMENT REGARDING FEDERALLY SPONSORED RESEARCH OR DEVELOPMENT

This invention was made with government support under AI154242 and AT010253 awarded by the National Institutes of Health. The government has certain rights in the invention.

### BACKGROUND

Over the last two decades, the therapeutic landscape of probiotics has expanded exponentially, with microbial supplementation linked to a broad spectrum of improved health outcomes ranging from obesity prevention to immune modulation. However, this proliferation of potential clinical application has largely outpaced our fundamental understanding of metabolic mechanisms that govern probiotic efficacy within the host niche. While the historical use of fermented microbes provides a dietary precedent, the translation to clinical therapeutics remains limited: currently only two microbial products (VOWST and REBYOTA) have achieved FDA approval as therapeutics specifically for recurrent C. difficile infection. The vast remainder of the market is dominated by supplements targeting gut, vaginal, or neurological health, which often lack the rigorous in vitro or clinical validation required to support their functional claims. This disconnect highlights a critical need to transition from empirical species selection toward a standardized functional rationale, enabling the rational design of next-generation live biotherapeutics for targeted dysbiosis. Accordingly, there is a need to address the aforementioned deficiencies and inadequacies.

### SUMMARY

Described herein are systems, methods, and computer readable media related to the identification of novel therapeutics, in particular, novel therapeutics for a dysbiotic vaginal microbiome that may lead to such conditions as symptomatic bacterial vaginosis. As would be understood from the present disclosure, any method involving any combination of steps can be stored as instructions on any non-transitory computer readable media or can be executed by any system as described herein.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1C: Survey of over-the-counter probiotic supplements. FIG. 1A) Histogram of the number of probiotic supplements containing a certain number of probiotic species. FIG. 1B) Box and whisker plot of the number of species contained in a probiotic supplement by brand-name. Only brand-names containing three or more unique products were displayed on this plot. Brand 1: n=43, min/max=1/15, median=4, Q1-Q3=1.5 to 8, Whiskers=1 to 15, P10/P25/P50/P75/P90=1/1.5/4/8/10. Brand 2: n=16, min/max=1/3, median=1, Q1-Q3=1 to 1, Whiskers=1 to 1, P10/P25/P50/P75/P90=1/1/1/1/2. Brand 3: n=17, min/max=1/15, median=12, Q1-Q3=2 to 14, Whiskers=1 to 15, P10/P25/P50/P75/P90=1.6/2/12/14/15. Brand 4: n=25, min/max=1/4, median=1, Q1-Q3=1 to 2, Whiskers=1 to 2, P10/P25/P50/P75/P90=1/1/1/2/2. Brand 5: n=9, min/max=1/2, median=2, Q1-Q3=1 to 2, Whiskers=1 to 2, P10/P25/P50/P75/P90=1/1/1/2/2. Brand 6: n=9, min/max=1/1, median=1, Q1-Q3=1 to 1, Whiskers=1 to 1, P10/P25/P50/P75/P90=1/1/1/1/1. Brand 7: n=36, min/max=1/9, median=3, Q1-Q3=1 to 4, Whiskers=1 to 8, P10/P25/P50/P75/P90=1/1/3/4/5. Brand 8: n=18, min/max=1/10, median=1.5, Q1-Q3=1 to 8.5, Whiskers=1 to 10, P10/P25/P50/P75/P90=1/1/1.5/8.5/10. Brand 9: n=9, min/max=1/11, median=9, Q1-Q3=2 to 9, Whiskers=1 to 11, P10/P25/P50/P75/P90=1.8/2/9/9/10.2. Brand 10: n=9, min/max=1/15, median=4, Q1-Q3=2 to 8, Whiskers=1 to 15, P10/P25/P50/P75/P90=1.8/2/4/10/11. FIG. 1C) PCA plot of the species profiles of each probiotic supplement. Colors of points represent the marketed use for each probiotic supplement.

FIGS. 2A-2H: Description of genome-scale metabolic network models of probiotic, pathogen, and host-associated bacterial metabolism. FIG. 2A) Workflow for generating the HaPaPro GENRE collection. FIG. 2B) Number of unique reactions across the GENREs of each category. FIG. 2C) Number of shared reactions between probiotic, pathogen, and host-associated organisms. FIGS. 2D-2F) Metabolic subsystems of reactions that are unique to host-associated (FIG. 2D), pathogen (FIG. 2E), and probiotic (FIG. 2F) GENREs. FIG. 2G) PCA of metabolic phenotypes of probiotic GENREs. Colors represent distinct taxonomic families. Triangles represent cluster centroid; ellipses plotted 2 standard deviations from the centroid. Kruskall-Wallis test for median distances between centroids, p-value=1.16e-112. Cluster standard deviations are as follows: Lactobacillus: 995.19, Streptococcaceae: 533.53, Bacillaceae: 635.82. Bifidobacteriaceae: 609.72, Akkermansiaceae: 248.95, and Enterococcaceae: 488.13. FIG. 2H) PCA of metabolic phenotypes across probiotic, pathogen, and host-associated GENREs. Triangles represent cluster centroid, ellipses plotted represent two standard deviations from the centroid. Kruskall-Wallis test for median distances between centroids, p-value<0.0001.

FIGS. 3A-3B: Functional analysis of vaginal probiotic species and other vaginal bacteria. FIG. 3A) PCA plot of metabolic phenotypes of vaginal bacteria and probiotic species. Triangles represent cluster centroids, ellipses plotted 2 standard deviations from the centroid. Kruskal-wallis test for median distances between centroids, p-value=2.2e-7. Vaginal bacteria standard deviation: 897.7. Vaginal probiotic standard deviation: 863.35. FIG. 3B) Hierarchical clustering of metabolic phenotypes across vaginal probiotic species, two G. vaginal strains, and other culturable vaginal bacterial species. Metabolic phenotype vectors are calculated as the per-reaction median value across 500 flux samples.

FIGS. 4A-4F: In vitro spent media assay and lactic acid quantification. FIG. 4A) Growth curves of G. vaginalis 14018 in 12 media conditions (11 spent media and PGY (Modified) control). Three growth dynamics are specified to the right of the growth curves. N: non-inhibitory (n=4), M: moderate (n=4), I: inhibitory (n=4). FIG. 4B) Quantification of area under the growth curve for each of the three growth dynamic categories. Pairwise t-tests, one tailed: N vs M: p-value=5.68e-4, N vs I: p-value=4.19e-7, M vs I: p-value=6.76e-6. FIG. 4C) pH of the spent media in each growth dynamic category. Pairwise t-tests, one tailed: I vs M: p-value=6.46e-4, I vs N: p-value=6.32e-4, M vs N: p-value=0.4039. FIG. 4D) L-lactic acid concentration in the spent media of each growth dynamic category. Pairwise t-tests, one tailed: I vs M: p-value=0.133, I vs N: p-value=0.0532, M vs N: p-value=0.1605. FIG. 4E) L-lactic acid concentration in the spent media of each growth dynamic category. Pairwise t-test, one tailed: I vs M: p-value=0.0292, I vs N: p-value=0.02418, M vs N: p-value 0.1632. FIG. 4F) Relationship between D-lactic acid and G. vaginalis AUC. R²=0.7026, exponential fit equation=y=122.504e^{-0.564x}. Bar chart significance key: ***=p<0.001, **=p<0.01, *=p<0.1, ns=not significant. All data are presented as mean values+/−standard deviation.

FIG. 5: Number of “Probiotics” PubMed articles published each year since 1950.

FIG. 6: In silico metabolic phenotype comparison between vaginal strains and G. vaginalis strains. Hierarchical clustering of metabolic phenotypes across two G. vaginalis strains, and culturable vaginal commensal species used in the in vitro spent media experiment, rows are colored by G. vaginalis growth dynamic category. Metabolic phenotype vectors are calculated as the per-reaction median value across 500 flux samples.

FIGS. 7A-7C: Comparison of culturable vaginal commensal species growth in four media conditions. Growth over 48 and 72 hour periods in PGY, PGY (Modified), NYCIII, and NYCIII (Modified). N=1.

FIG. 8: In silico metabolic phenotype comparison between vaginal strains and G. vaginalis strains. Hierarchical clustering of metabolic phenotypes across two G. vaginalis strains, and culturable vaginal commensal species used in the in vitro spent media experiment, rows are colored by G. vaginalis growth dynamic category. Metabolic phenotype vectors are calculated as the per-reaction median value across 500 flux samples.

FIGS. 9A-9C: G. vaginalis grown in co-culture with high D-lactate producing bacteria. Growth curve results of G. vaginalis grown in co-culture with L. jensenii (FIG. 9A), A. lactolyticus (FIG. 9B), and A. tetradius (FIG. 9C) over a 16-hour time course. All growth curve data presented here are from one replicate (n=1). 1:10 ratio represents a ratio of OD 0.005:0.05 where 0.005 is a 1:10 dilution from a culture with an OD of 0.05.

FIG. 10 is a is a block diagram illustrating an example of a machine upon which one or more aspects of embodiments of the present disclosure can be implemented.

FIG. 11 is a flowchart illustrating one example of a method according to various embodiments of the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of genetics, microbiology, biochemistry, molecular biology, cellular biology, tissue culture, and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### DEFINITIONS

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit, or other things, for example, code to execute steps of methods as described herein in conjunction with a computing device. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “bacterial population” or “bacterial consortium” as used herein refers to a homogeneous population of bacteria consisting of a single strain of a bacterial species or a heterogeneous population of bacteria consisting of at least two distinguishable strains of a single bacterial species or of a plurality of species.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

The term “preventing” means to stop or hinder a disease, disorder, or symptom of a disease or condition through some action.

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects. These probiotic features are strain-dependent, even among bacteria of the same species. Therefore, it is important to find those strains that have a better performance in all probiotic requirements.

The term “reducing” means to diminish in extent, amount, or degree.

The term “therapeutic agent” as used herein refers to a bacterial probiotic, for example, comprising one or more bacterial species that can metabolically compete with bacterial causing symptoms of a bacterial infection.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

### Discussion

Described herein are systems, methods, and computer readable medium for, among other things, predictive platforms for novel probiotic therapeutics. Such systems, methods, and computer readable medium can be utilized for identifying novel bacterial species that can, for example, be utilized as therapeutics for administration against a candidate bacterial infection. In an embodiment, a candidate bacterial infection is Bacterial Vaginosis (BV). An aspect of the present disclosure also provides a system, method and computer readable medium for, among other things, algorithms/models that enabled identification of novel probiotics.

Many probiotics on the market that are marketed for a given condition are largely ineffective as the bacteria strains of the probiotic may not be associated with or otherwise improve the actual condition. Practical applications of methods described herein allow for the selection of improved probiotics that have a metabolic phenotype, for example, that can overlap with required resources of a given pathogen. The methods described herein thereby provide for selection of probiotics that can be tailored to a given pathological condition based specifically on one or more metabolic phenotypes that compete specifically for resources that are required by a given pathological bacterial strain. Such tailored competitive probiotics seek to improve the performance and efficacy of probiotics and reduce the impact of bacterial infection in a subject, when administered instead of a generic and untailored probiotic.

### I. PLATFORMS FOR THERAPEUTIC TARGET IDENTIFICATION

Described herein are methods, systems, platforms, and machine-readable media (comprising machine-readable instructions for execution on a computing device or system described herein) relating to platforms to identify novel therapeutics (probiotics, for example) for the treatment of various disorders, for example, a bacterial infection such as symptomatic or asymptomatic BV. In embodiments, the methodologies and platforms are related to novel therapeutics for BV, novel probiotics for example.

### A. Methodologies

Described herein are methods of identifying candidate therapeutics and therapeutic targets against a candidate bacterial infection, for example, probiotics. Through metagenomic sequencing and metabolomic analysis, probiotic bacterial populations that cooperate, compete, or otherwise effect net mass flux with one or more species of pathological bacteria can be identified.

In examples, described herein are methods of identifying candidate probiotics for administration against a candidate bacterial infection.

In examples, a method of identifying candidate probiotics for administration against a candidate bacterial infection can comprise identifying one or more candidate probiotic species, identifying one or more candidate pathogenic bacterial species (for examples, ones that are symptomatic and cause one or more symptoms of a bacterial infection), and identifying one or more candidate host bacterial species.

In examples, methods further comprise selecting a representative genome of each of the one or more candidate probiotic bacterial species, one or more of candidate host bacterial species, and one or more candidate host bacterial species.

In examples, methods further comprise generating an annotated protein sequence for each representative genome by assigning functional pathways to a set of proteins encoded by each representative genome.

In examples, methods further comprise determining superorganism-level functional metabolic capacity from each of the annotated protein sequences of each of the one or more candidate probiotic bacterial species, one or more of candidate host bacterial species, and one or more candidate host bacterial species.

In examples, methods further comprise identifying metabolic reactions with differential metabolic capacities from the determined superorganism-level functional metabolic capacities between the one or more candidate probiotic bacterial species, one or more candidate pathogenic bacterial species, and one or more candidate host bacterial species.

In examples, methods further comprise clustering the identified metabolic reactions with differential metabolic capacities according to a metabolic subsystem. In examples, clustering the identified metabolic reactions with differential metabolic capacities according to a metabolic subsystem can be optional.

In examples, methods further comprise characterizing the metabolic phenotype from the identified metabolic reactions with differential metabolic capacities from each of the representative genomes of each of the one or more candidate probiotic bacterial species, one or more of candidate host bacterial species, and one or more candidate host bacterial species.

In examples, methods further comprise clustering the characterized metabolic phenotypes.

In examples, methods further comprise selecting one or more probiotic species from the one or more candidate probiotic bacterial species according to clustered characterized metabolic phenotypes that would metabolically compete with the one or more candidate pathogenic bacterial species.

In examples, methods further comprise selecting one or more probiotic species from the one or more candidate probiotic bacterial species according to clustered characterized metabolic phenotypes that would metabolically cooperate with the one or more candidate host bacterial species. In examples, the metabolic phenotypes that provide for metabolic cooperation of the one or more candidate probiotic bacterial species and one or more candidate host bacterial species can metabolically compete with the one or more candidate pathogenic bacteria.

In examples, the one or more candidate pathogenic bacterial species can be identified from an anatomical area of a subject to infection or a biological sample. In examples, the identification can be with provided metagenomic sequencing data of the one or more candidate pathogenic bacterial species or obtaining metagenomic sequencing data of the one or more candidate pathogenic bacterial species. In examples, a taxonomic bacterial composition of the one or more candidate pathogenic bacterial species can be obtained from the metagenomic sequence data.

In examples, the one or more candidate pathogenic bacterial species can cause a Bacterial Vaginosis (BV) diagnosis in a subject. In examples, the one or more candidate host bacterial species are identified from a vaginal swab from one or more subjects having or suspected of having Bacterial Vaginosis (BV). In examples, the one or more candidate pathogenic bacterial species comprise a G. vaginalis strain.

In examples, the selecting can be by selecting a representative reference sequence from a database, or, if none exists, randomly selecting a representative sequence that is considered good and complete.

In examples, the superorganism-level functional capacity can be determined by mapping metagenomic reads of each representative genome to the annotated proteins and differentiating functionalities of each mapped metagenomic read.

In examples, determining superorganism-level functional metabolic capacity can be with genome-scale metabolic network reconstructions (GENREs). In examples, each superorganism-level functional metabolic capacity can be determined with a genome scale metabolic network reconstructions. In examples, any aspects of methods described herein can be with data from genome-scale metabolic network reconstructions (GENREs) derived from a representative genome.

In examples, identifying metabolic reactions with differential metabolic capacities according to a metabolic subsystem comprises annotating the gene content of each cluster, quantifying the annotated gene content, segregating the quantified gene content based on one or more candidate probiotic bacterial species, one or more of candidate host bacterial species, and one or more candidate host bacterial species; identifying genes enriched in one or more metabolic pathways from the segregated quantified gene content of the one or more candidate probiotic bacterial species compared to the one or more of candidate host bacterial species and the one or more candidate host bacterial species; and reconstructing the metabolism of the one or more metabolic pathways from the quantified gene content with gene identities mapped onto the one or more candidate probiotic bacterial species, the one or more of candidate host bacterial species, and the one or more candidate host bacterial species

In examples, a metabolic subsystem comprises one or more of glycan, secondary metabolites, energy, terpenoids and polyketides, xenobiotics, nucleotide, lipid, cofactors and vitamins, carbohydrate, or amino acid.

In examples, characterizing the metabolic phenotype is with flux balance analysis (FBA) and/or flux space sampling.

In examples, characterizing the metabolic phenotype comprises segregating the reconstructed metabolism of each representative genome into cooperative or competitive secondary candidate bacterial species in relation to the one or more candidate pathogenic bacterial species.

In examples, a method can further comprise culturing the one or more selected probiotic bacterial species in the presence of cell culture media to create spent probiotic media comprising metabolic byproducts derived from the one or more selected probiotic bacterial species, culturing the one or more identified candidate pathogenic bacterial species separately from the one or more selected probiotic bacterial species, administering the spent probiotic media to the one or more identified candidate pathogenic bacterial species cultures, and determining a level of growth of the one or more identified candidate pathogenic bacterial species in the presence of the spent probiotic media.

In examples, a method further comprises culturing the one or more selected host bacterial species in the presence of cell culture media to create spent host media comprising metabolic byproducts derived from the one or more selected host bacterial species, and administering the spent host media to the one or more identified candidate pathogenic bacterial species cultures before, during, or after the administration of the spent probiotic media, determining a level of growth of the one or more identified candidate pathogenic bacterial species in the presence of the spent probiotic media and spent host media.

In examples, the one or more selected probiotic bacterial species and one or more host bacterial species are co-cultured to create a spent mixed media comprising metabolic byproducts of both the one or more selected probiotic bacterial species and one or more host bacterial species.

In examples, a method can comprise determining levels of one or more metabolic byproducts in the spent media. In examples, one or more metabolic byproducts comprise D-lactic acid, L-lactic acid, or both.

In examples, a method further can comprise measuring the pH of the spent media.

In embodiments, samples collected and analyzed, or the source of provided data, are derived from vaginal swabs (i.e., microbiota from the vagina of a subject that is collected in a standard swab).

In embodiments, methods as described herein can further comprise sequencing the metagenome of the one or more biological samples (e.g., vaginal swabs of a subject as discussed above) of the host, biological sample, or an anatomical region of the hose. In embodiments, the metagenomic sequencing comprises next-gen sequencing.

Methods as described herein can comprise determining superorganism-level functional metabolic capacity from the metagenomic sequencing data of the probiotic, pathogenic, host group. In embodiments, the superorganism-level functional capacity can be determined by assembling contiguous genomic sequences from the metagenomic sequencing data; annotating gene content by assigning functional pathways to the assembled genomic sequences; mapping metagenomic reads to annotated genes; and differentiating functionalities of the mapped metagenomic reads.

Methods as described herein can comprise identifying metabolic pathways with differential metabolic capacities between the groups (probiotic, pathogenic, host) from the determined capacities.

Methods as described herein can comprise clustering the groups (probiotic, pathogenic, host) based on the identified determined functional metabolic capacities.

Methods as described herein can comprise annotating the gene content of each cluster.

Methods as described herein can comprise quantifying the annotated gene content.

Methods as described herein can comprise segregating the quantified gene content based on bacterial type (i.e., candidate probiotic, candidate pathogenic, or host).

Methods as described herein can comprise identifying genes enriched in one or more metabolic pathways from the segregated quantified gene content.

Methods as described herein can comprise reconstructing the metabolism of the one or more metabolic pathways mapped onto one or more species of the probiotic, host, and/or pathogenic bacteria.

Methods as described herein can comprise segregating the reconstructed metabolism of the bacterial species into cooperative or competitive with the candidate bacterial infection species. In embodiments, segregating the quantified gene content is using machine learning. Machine learning can be utilized and/or implemented according to methods known in the art. In embodiments, the machine learning is supervised, semi-supervised, unsupervised or reinforcement. In embodiments, the machine learning utilizes random forests, support vector machines. In embodiments, the machine learning utilizes classification, regression, and/or clustering.

In embodiments, reconstructing the metabolism and segregating the reconstructed metabolism is with genome-scale metabolic network reconstructions (GENREs). GENREs are comprehensive mathematical representations of metabolic reactions in a cell. GENREs include the mapping of gene annotation to reaction stoichiometry and capture the stoichiometry as a mathematical matrix. Additional information relating to GENREs can be found, for example, in Bauer, E. & Thiele, I. From Network Analysis to Functional Metabolic Modeling of the Human Gut Microbiota. mSystems 3, e00209-17 (2018); Bauer, E. & Thiele, I. From metagenomic data to personalized in silico microbiotas: predicting dietary supplements for Crohn's disease. NPJ Syst Biol Appl 4, 27 (2018); and Jenior, M. L. et al. Novel Drivers of Virulence in Clostridioides difficile Identified via Context-Specific Metabolic Network Analysis. mSystems 6, e0091921 (2021), the entire contents of all of three of which are incorporated by reference as if fully set forth herein. Additional details of GENRE's as described herein can be found, for example, in Jenior, M. L., Leslie, J., Kolling, G. L., Archbald-Pannone, L., Powers, D., Petri, W. A., & 578 Papin, J. A. (2023). Systems-ecology designed bacterial consortium protects from severe Clostridioides difficile infection. bioRxiv, which is incorporated by reference as if fully set forth herein regarding methods of GENRE construction and GENREs utilized according to methods and systems of the present disclosure.

Methods as described herein can comprise identifying one or more bacterial species for administration having cooperative or competitive metabolism with the candidate bacterial infection.

In embodiments of methods as described herein, the one or more metabolic pathways are one or more of amino acid biosynthesis, combined galactose metabolism, amino sugar metabolism, nucleotide sugar metabolism, starch metabolism, or sucrose metabolism, individually or in any combination thereof.

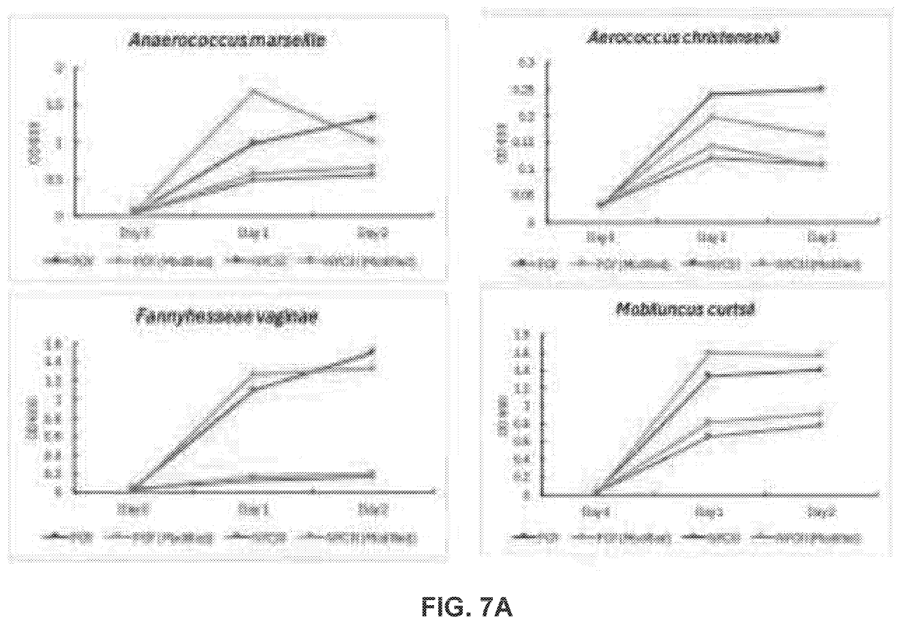

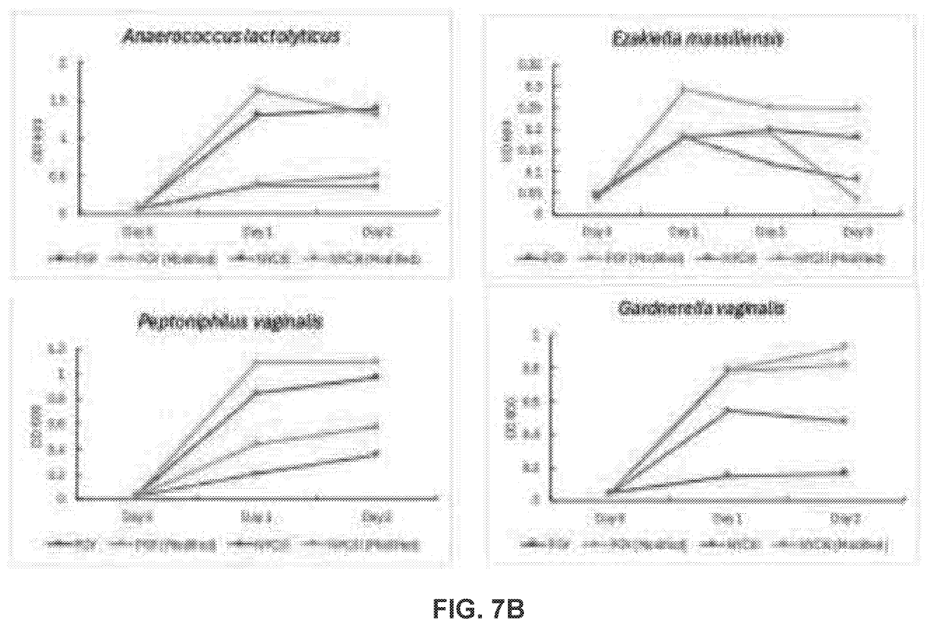

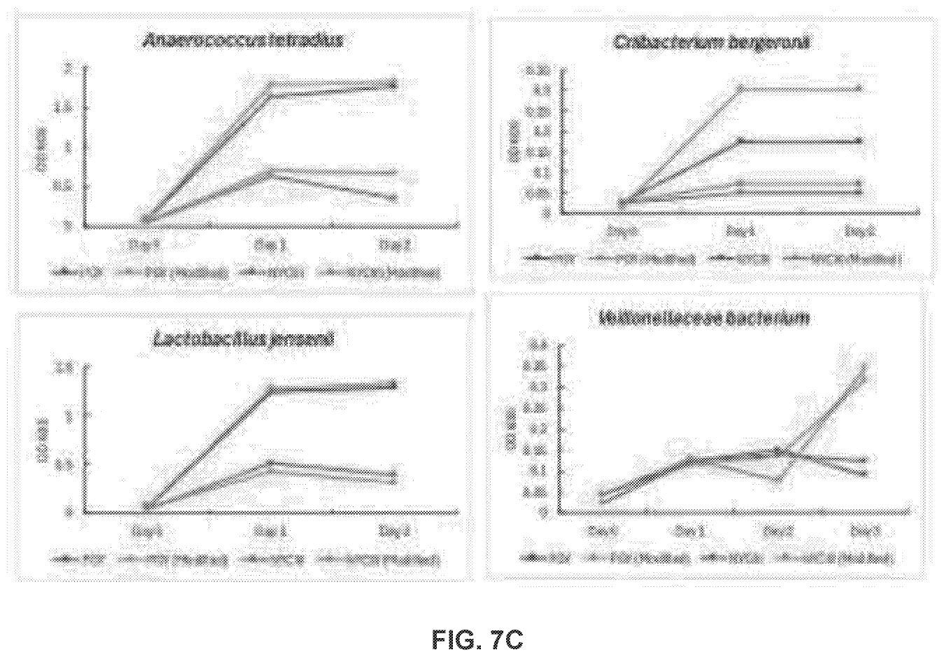

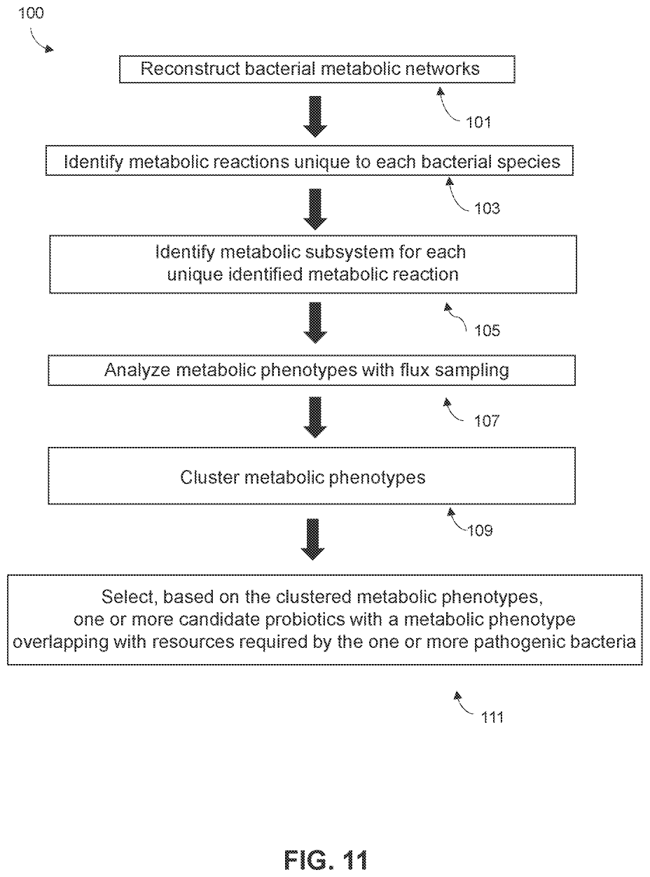

Referring next to FIG. 11, shown is a flowchart that provides an example of the operation of aspects of methods as described herein, method 100. The flowchart of FIG. 7 provides merely an example of the many different types of functional arrangements that can be employed to implement the operation of the depicted portion of methods described herein. As an alternative, the flowchart of FIG. 7 can be viewed as depicting an example of elements of a method implemented within a local system or a network environment as discussed herein.

Beginning with block 101, the method can reconstruct metabolic networks from one or more representative genomes of each of one or more candidate probiotic bacterial species, one or more candidate host bacterial species, and one or more candidate host bacterial species. In examples, this step of block 101 can also comprise generating an annotated protein sequence for each representative genome by assigning functional pathways to a set of proteins encoded by each representative genome. In examples, this step of block 101 can further comprise determining superorganism-level functional metabolic capacity from each of the annotated protein sequences of each of the one or more candidate probiotic bacterial species, one or more of candidate host bacterial species, and one or more candidate host bacterial species.

Next, with block 103, metabolic subsystem for each unique identified metabolic reaction can be identified. In examples, block 103 can comprise identifying metabolic reactions with differential metabolic capacities from determined superorganism-level functional metabolic capacities between the one or more candidate probiotic bacterial species, one or more candidate pathogenic bacterial species, and one or more candidate host bacterial species. In examples, block 103 can comprise clustering the identified metabolic reactions with differential metabolic capacities according to a metabolic subsystem.

In block 105, metabolic subsystems for each unique identified metabolic reaction can be identified. In examples, block 105 can further comprise clustering the identified metabolic reactions with differential metabolic capacities according to a metabolic subsystem.

In block 107, metabolic phenotypes can be analyzed with flux sampling. In examples, block 107 can further comprise characterizing the metabolic phenotype from the identified metabolic reactions with differential metabolic capacities from each of the representative genomes of each of the one or more candidate probiotic bacterial species, one or more of candidate host bacterial species, and one or more candidate host bacterial species.

In block 109, metabolic phenotypes can be clustered. In examples, the identified unique metabolic reactions can be clustered according to differential metabolic capacities compared to the one or more pathogenic bacterial species.

In block 111, one or more candidate probiotics can be selected that have a metabolic phenotype overlapping with resources required by the one or more pathogenic bacteria. In examples, the metabolic phenotype comprises unique metabolic reactions that are not observed in the one or more host bacteria and/or one or more pathogenic bacteria. In examples, the unique metabolic reactions may also have differential activity compared to other bacteria.

The flowchart[s] and sequence diagrams show the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowcharts and sequence diagrams show a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowcharts and sequence diagrams can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

### B. Computer Readable Media and Systems for Predictive Probiotic Platforms

Described herein are computer-readable medium, comprising machine-readable instructions stored in the memory that, when executed by a processor of a computing device, cause the computing device to at least carry out a method as described herein.

Described herein are systems, comprising, for example, a computing device comprising a processor and a memory, and machine-readable instructions stored in the memory that, when executed by the processor, cause the computing device to at least carry out a method as described herein.

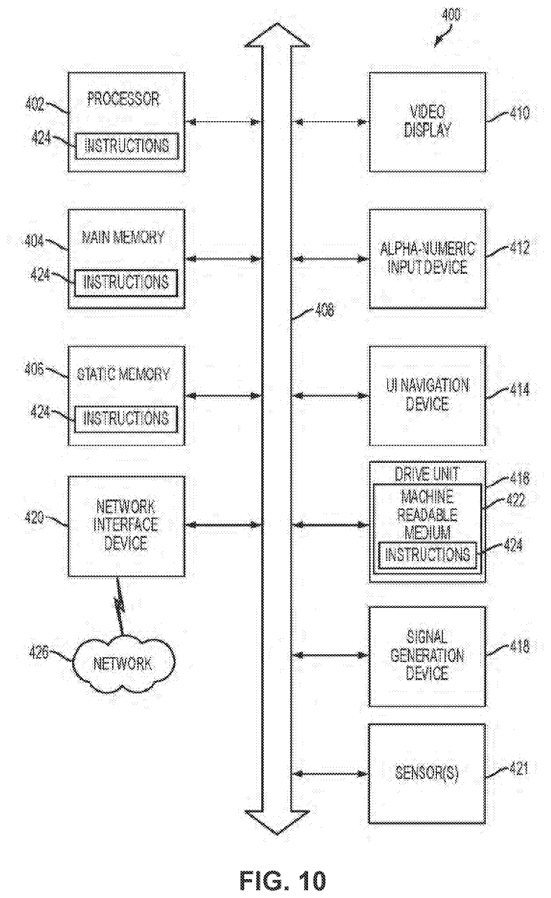

FIG. 10 is a block diagram illustrating an example of a machine or system upon which one or more aspects of embodiments of the present invention can be implemented (in particular, systems and machine-readable media that can perform the methods in (A) above).

Referring to FIG. 10, an aspect of an embodiment of the present invention includes, but not limited thereto, system, method and computer readable medium for, among other things, carrying out one or more methods described herein. A block diagram of an example machine 400 upon which one or more embodiments (e.g., discussed methodologies) can be implemented (e.g., run) is shown in FIG. 10. Such systems described herein can be utilized to run any of the methods described herein, in particular, those embodied by the examples and as described in the methodologies above.

Examples of a machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between suet, circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., witt1in a t1ome environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a standalone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that tt1e choice of whether to implement certain functionality in permanently configured t1ardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer mact1ine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The mact1ine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### II. MICROBIAL POPULATIONS

Samples comprising microbial compositions can be analyzed according to methods and systems of the present disclosure. Samples as described herein can be biological samples taken from a subject that comprise one or more bacterial strains (also referred to as “microbiota”). In embodiments, the one or more bacterial strains can comprise bacteria (also referred to herein as “micro-organism” or “micro-organisms”) that can be gram-positive, nonmotile, branched anaerobic bacteria. In embodiments, the one or more bacterial strains can comprise bacteria that can be gram-negative, facultative anaerobic, rod-shaped, coliform bacteria. In embodiments, the one or more bacterial strains can comprise bacteria that can be gram-positive anaerobic bacteria. In embodiments, the one or more bacterial strains can comprise bacteria that can be gram-positive and facultatively anaerobic. Bacteria consortia as described herein comprise viable and metabolically active micro-organisms, which can be proliferative or non-proliferative.

In embodiments, one or more host bacterial species can be cultured and/or isolated from a biological sample from a subject, in particular an anatomical area of a subject, that is not experiencing symptoms of a bacterial infection (i.e., a healthy or control subject). In embodiments, one or more host bacterial species can be cultured and/or isolated from a biological sample from a subject, in particular an anatomical area of a subject, that is experiencing symptoms of a bacterial infection at the time the sample is taken (i.e., a subject having an infection). For the subject having the infection, the sample can be taken from an anatomical area of the body experiencing symptoms, for example, a vaginal swab from an individual experiencing symptoms of Bacterial vaginosis (BV).

Pathogenic samples according to the present disclosure, in certain aspects, may be enriched in L. iners, P. amnii, P. buccalis, H. timonensis, F. vaginae, A. christensii, G. vaginalis, and G. piotii, thereby containing these strains at a higher level than other strains. Samples as described herein may, as a primary bacterial candidate, comprise Gardnerella species, as well as others as described herein.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### III. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in °C, and pressure is in atmosphere. Standard temperature and pressure are defined as 25 °C and 1 atmosphere.

### Example 1

Probiotic supplements are marketed for diverse health benefits, yet species inclusion often lacks functional rationale. The present example tested a sample of 352 over-the-counter probiotic products available in the US and revealed 36 unique microbial species. However, there is no clear link between species inclusion and the intended health benefit. To address this gap, HaPaPro was developed, a collection of 1,012 genome-scale metabolic models spanning pathogenic, probiotic, and host-associated bacteria, constructed from publicly available genome sequences. Flux balance analysis revealed that probiotic species fail to capture the metabolic diversity of host-associated microbes. Focusing on vaginal health, vaginal microbes with metabolic profiles overlapping Gardnerella vaginalis were computationally identified. In vitro spent media assays using 11 vaginal isolates showed variable inhibition of G. vaginalis, primarily driven by D-lactic acid production which was also produced by non-Lactobacillus species. These findings highlight the need for function-based probiotic design and demonstrate a scalable framework integrating metabolic modeling with experimental validation

### A. Introduction

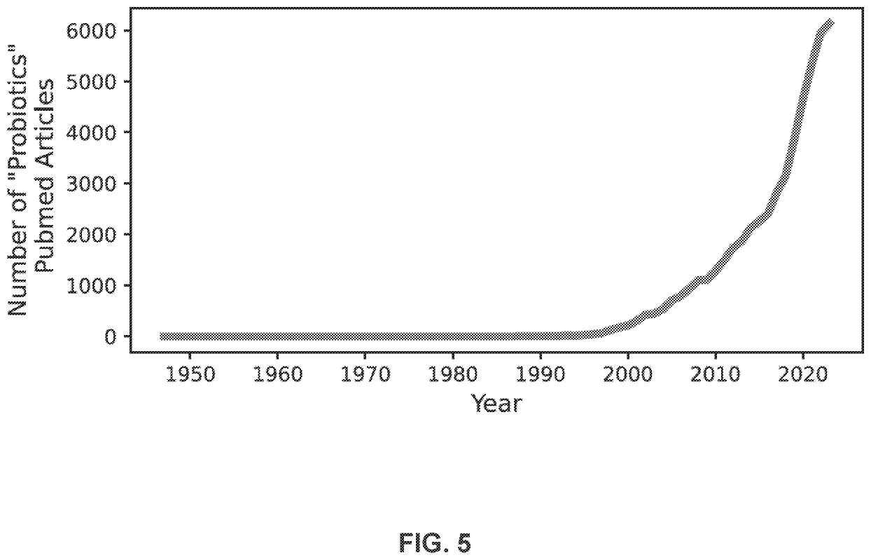

Over the last two decades, the therapeutic landscape of probiotics¹ has expanded exponentially (FIG. 5), with microbial supplementation linked to a broad spectrum of improved health outcomes ranging from obesity prevention to immune modulation²⁻⁵. However, this proliferation of potential clinical application has largely outpaced our fundamental understanding of metabolic mechanisms that govern probiotic efficacy within the host niche. While the historical use of fermented microbes provides a dietary precedent⁶, the translation to clinical therapeutics remains limited; currently only two microbial products (VOWST and REBYOTA^{7,8}) have achieved FDA approval as therapeutics specifically for recurrent C. difficile infection. The vast remainder of the market is dominated by supplements targeting gut, vaginal, or neurological health, which often lack the rigorous in vitro or clinical validation required to support their functional claims^{9,10}. This disconnect highlights a critical need to transition from empirical species selection toward a standardized functional rationale, enabling the rational design of next-generation live biotherapeutics for targeted dysbiosis.

Women's health represents a primary frontier for probiotic development, with the vaginal microbiome serving as a critical niche for ecological intervention¹¹⁻¹⁴. In reproductive-age women, homeostasis is typically maintained by Lactobacillus dominance and low pH (pH<4.5), which provides a robust barrier against pathogen colonization¹⁵. Conversely, the dysbiotic state of bacterial vaginosis (BV) is characterized by a significant rise in pH (>4.5), an increase in taxonomic diversity, and the proliferation of anaerobic pathobionts such as Gardnerella species¹⁶⁻¹⁸. While antibiotics remain the clinical standard, high recurrence rates^{19,20} and the rise of antimicrobial resistance underscore the limitations of these broad-spectrum approaches²¹.

Critically, recent evidence has identified healthy, asymptomatic populations with naturally diverse microbiomes that exist in a dynamic equilibrium with both Gardnerella and Lactobacillus²². This heterogeneity suggests that health may be governed by functional niche stability rather than simple species exclusion, challenging the efficacy of “one-size-fits-all” treatments. There is a pressing need for a patient-specific framework that accounts for these demographic variations utilizing rationally designed probiotics to stabilize the metabolic landscape and prevent pathogen establishment across diverse host environments^{16,19,20,22}.

Microbial restoration through oral or vaginal probiotic administration offers a compelling strategy for re-establishing vaginal homeostasis^{23,24}. To contextualize the efficacy of current interventions, 32 independent research studies treating recurrent BV with probiotics, antibiotics or combination therapies²⁵⁻⁵⁵ were surveyed. Across these studies, there was no clear link between treatment modality and durable protection against BV recurrence. However, current clinical interventions have yielded highly variable results, with existing formulations frequently failing to provide durable protection against BV recurrence compared to standard antibiotic regimens^{37,41,50}. This inconsistency likely reflects the underlying ecological and demographic heterogeneity of the vaginal niche, suggesting that a “one-size-fits-all” approach to strain selection is inadequate. These observations highlight a critical opportunity for the development of personalized, rationally designed biotherapeutics. By shifting focus toward the specific metabolic mechanisms required to inhibit Gardnerella colonization, development of tailored probiotic consortia that provide robust and enduring support for vaginal health across diverse patient populations is possible.

In this study, market analysis, systems modeling, and in vitro experimentation is integrated to establish a functional rationale for probiotic design. First, the current market landscape is characterized through an extensive survey of products from the three largest U.S. pharmacies. To evaluate the metabolic capacity of these species, genome-scale metabolic network reconstructions (GENREs) and constraint-based reconstruction and analysis (COBRA)⁵⁶ methods are utilized to simulate the strain-specific phenotypes of 1,012 probiotic, pathogenic, and host-associated bacteria. This computational framework, termed the HaPaPro collection, enables the identification of unique metabolic signatures and significant functional gaps in current probiotic formulations.

Focusing on the vaginal microbiome as a clinical case study, these in silico simulations are combined with in vitro spent media and co-culture assays to identify native species that effectively inhibit Gardnerella vaginalis. By elucidating the shared metabolic traits and inhibitory mechanisms of these successful competitors, a mechanistic blueprint is provided for the rational development of targeted biotherapeutics capable of preventing vaginal dysbiosis.

### B. Results

### Probiotic Supplement Market Survey

To gain a deeper understanding of the landscape of over-the-counter probiotic supplements and the diversity of bacterial species they contain, a survey of all over-the-counter probiotic supplements available at the top three pharmacies in the United States was performed⁵⁷. Across these pharmacies there were 352 unique probiotic supplement products available for purchase, 36 unique probiotic species across these products, and 70 distinct brand names.

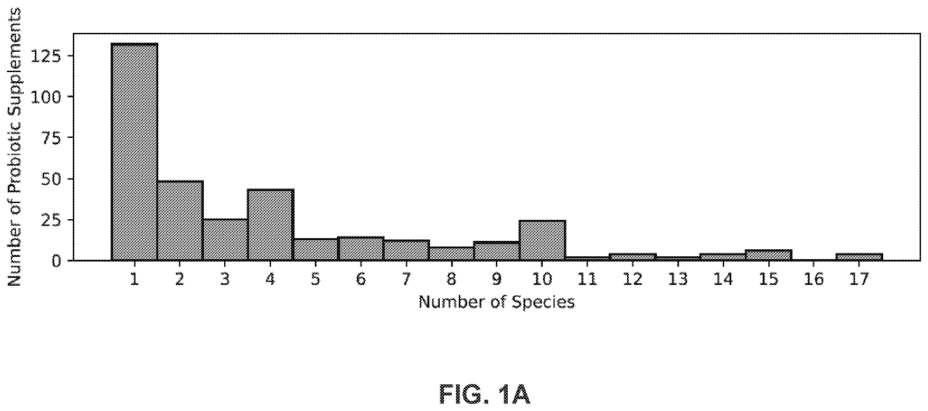

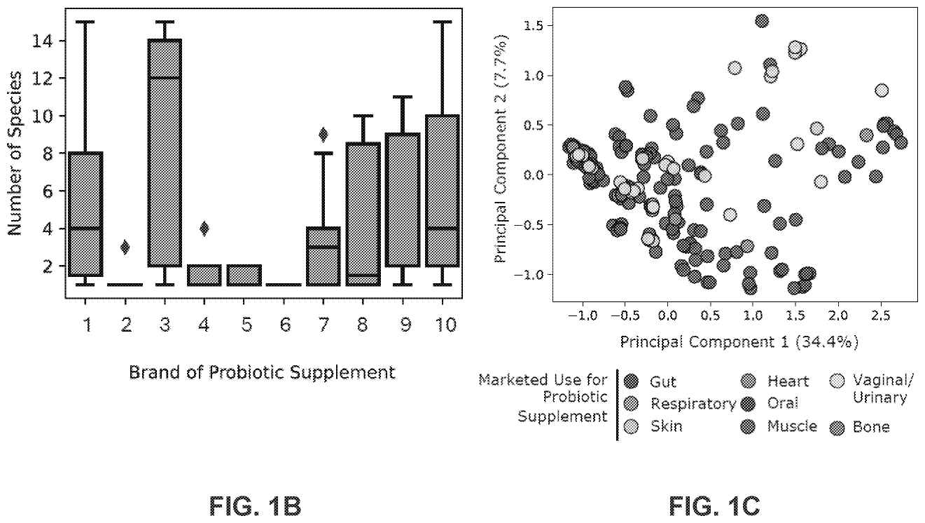

The 10 most prevalent species of bacteria found across the 352 probiotic supplements are detailed in Table 1 below. The top two species L. rhamnosus and L. acidophilus, both lactic acid producing bacteria, were present in 46% of all probiotic supplements. Additionally, B. coagulans was present in 22% of probiotic supplements. It is noted that over half of the probiotic supplements contain one probiotic species (FIG. 1A). Despite multi-strain probiotics being more effective at displacing pathogens due to synergy across strains⁵⁸, there are only several supplements that contain more than five unique species, and several that contain up to 17 unique species; perhaps reflecting industrial and manufacturing constraints rather than biological efficacy.

TABLE 1
Survey of Over-the-Counter Probiotic Supplements. Top 10 Most
Prevalent Probiotic Species Across Surveyed Supplements.

| | | % of All |
|---|---|---|
| Top 10 Most | Number of | Probiotic |
| Prevalent | Probiotic | Products |
| Species | Supplements | Surveyed |
| L. rhamnosus | 162 | 46% |
| L. acidophilus | 162 | 46% |
| B. longum | 103 | 29% |
| L. plantarum | 101 | 26% |
| B. lactis | 100 | 26% |
| L. paracasei | 90 | 26% |
| B. bifidum | 82 | 23% |
| B. coagulans | 79 | 22% |
| L. casei | 60 | 17% |
| B. breve | 59 | 17% |

Because of the variation in number of unique probiotic species included in a supplement, it was explored if the number of unique species was linked with brand-name (FIG. 1B). Evaluation of formulation complexity by brand revealed significant inter- and intra-brand variation, with species counts per product ranging from 1 to 17. While some brands maintain consistent species numbers across their product lines, other exhibit a broad distribution of in formulation diversity.

The targeted use for supplement along was identified with the profile of species either present or absent in each supplement. There was observed no apparent clustering in this PCA plot based on marketed use for the probiotic (FIG. 1C). This result suggests that across all over-the-counter probiotic supplements, there is no specific combination of species that are consistently used to specifically support gut health, vaginal health, etc. This result highlights the gap in understanding the functional capabilities of individual probiotic species.

### Metabolic Modeling of Diverse Bacterial Species

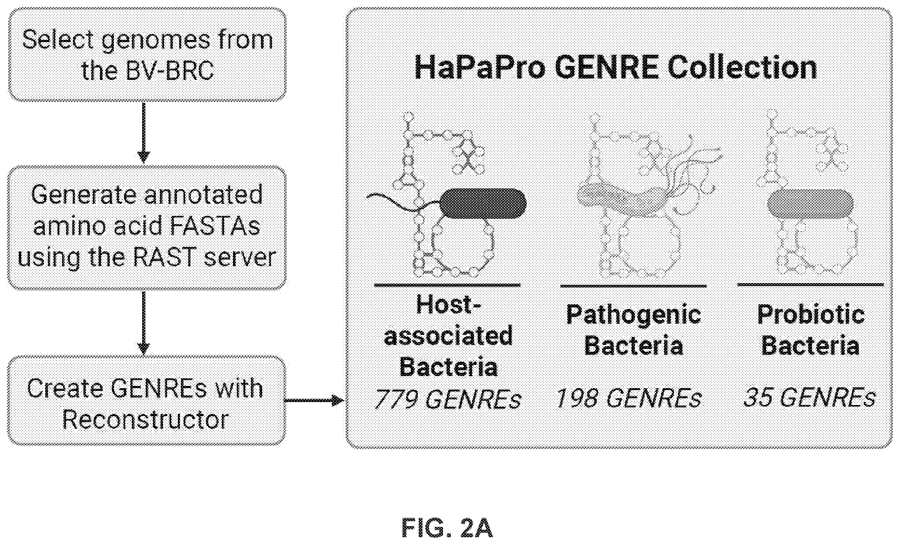

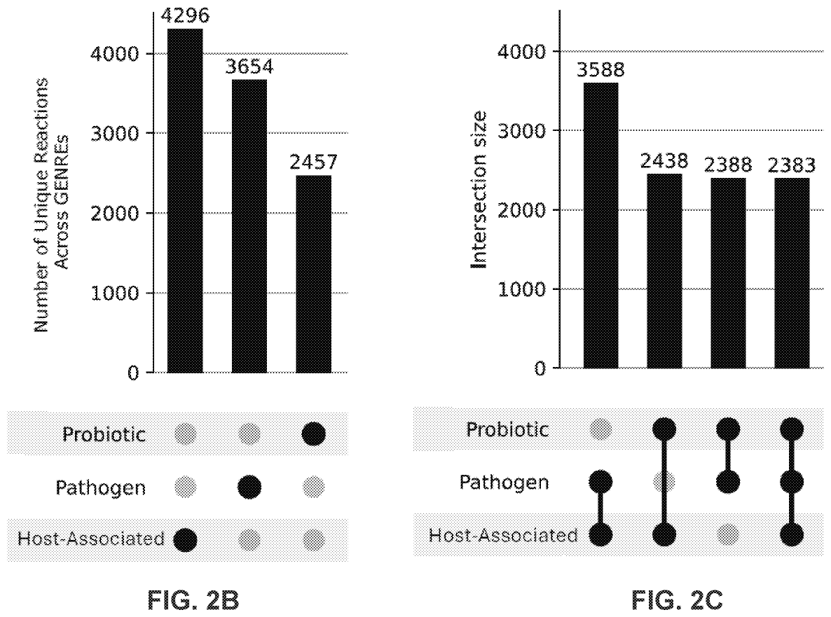

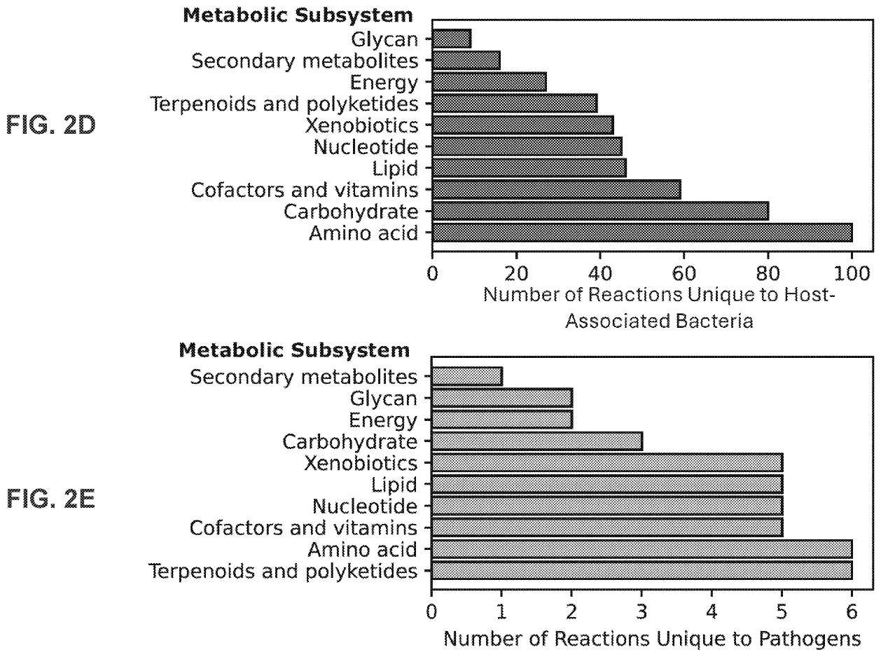

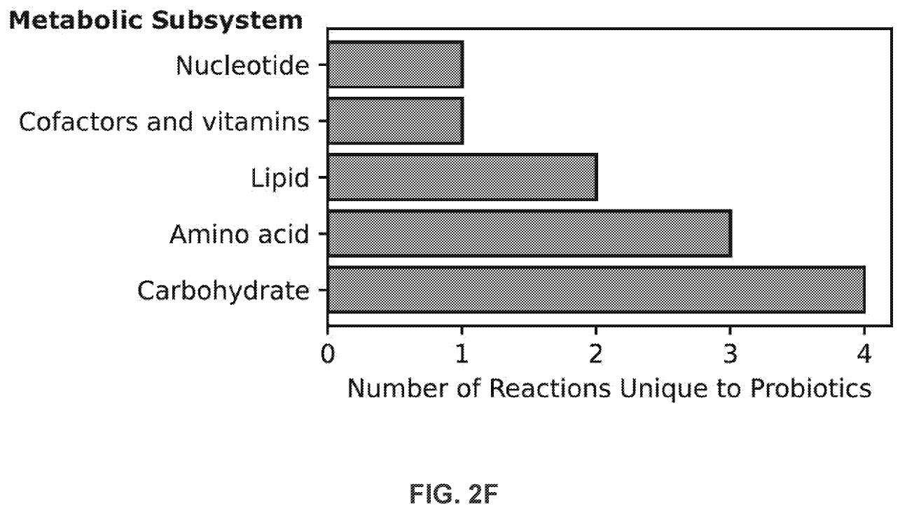

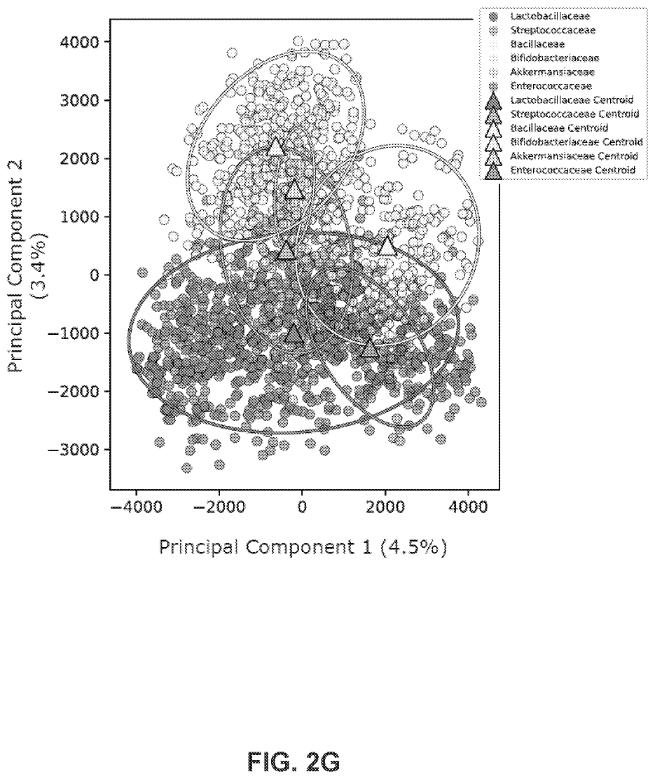

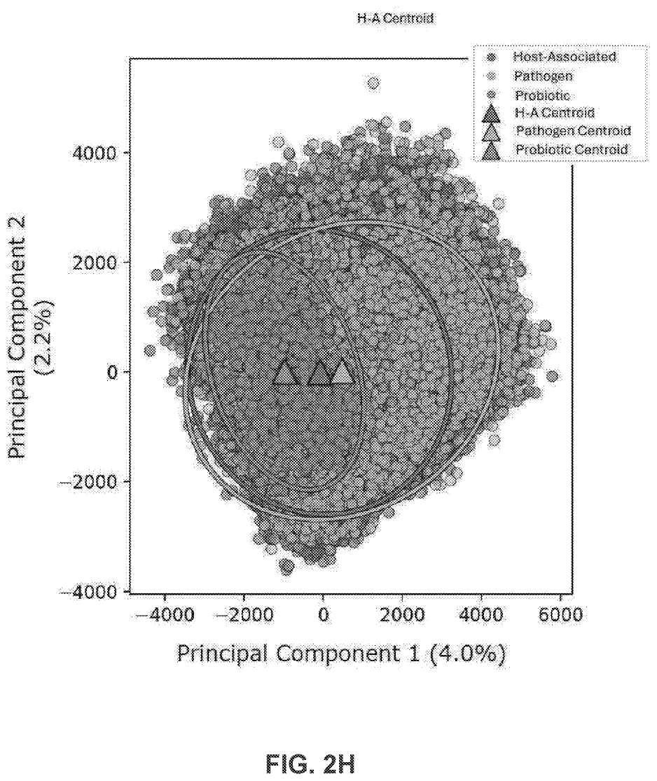

Genome-scale metabolic network reconstructions (GENREs) were generated representing 1,012 bacterial species using the automated GENRE construction tool, Reconstructor⁵⁹ (FIG. 2A, sequence selection outlined in methods) to assess the functional capabilities of probiotic, pathogenic, and other host-associated bacterial species—the HaPaPro collection. To systematically categorize this functional space probiotics were identified as species identified in a commercial market survey, pathogens as species included in the established PATHGENN database, and host-associated microbes as any remaining human-associated bacterial taxa in the BV-BRC database not captured by the prior two categories. No manual curation steps were performed with this collection due to the high quality of the draft reconstructions created with Reconstructor, and the number of reconstructions in the collection. With these GENREs, reaction content was explored across probiotic, pathogenic and other host-associated species.

Host-associated bacterial species have the greatest number of unique reactions across GENREs (4,296 reactions) likely due to category size and diversity of bacterial species it contains (FIG. 2B). Probiotics have the fewest unique reactions (2,457 reactions), likely due to the homogeneity of species the probiotic category contains (Lactobacillus, Bacillus, Bifidobacteria). There is significant overlap in reaction content between the three groups, with pathogens and non-pathogens sharing the greatest number of reactions (3,588) (FIG. 2C).

Across unique reaction sets, there were differences in reaction metabolic subsystems. Unique reaction sets were partitioned by group: host-associated pathogens were enriched in amino acid metabolism (FIG. 2D); host-associated bacteria are essential for the synthesis and absorption of certain amino acids in the human body^{60,61}. Pathogens were enriched in terpenoid and polyketide biosynthesis (FIG. 2E); which are responsible for the production of virulence factors in pathogens^{62,63}. Probiotics were enriched in carbohydrate metabolism (FIG. 2F); a result consistent with previous studies showing that carbohydrate metabolism in probiotic bacteria aids in digestion and promotes short chain fatty acid synthesis⁶⁴⁻⁶⁷. This metabolic heterogeneity confirms that host-associated, pathogen, and probiotic categories occupy distinct functional niches⁶⁸.

### Simulation of Metabolic Niche Differences

The GENREs of pathogenic, probiotic, and host-associated bacterial metabolism from the HaPaPro collection were utilized to probe each species' genotype-phenotype relationship using constraint-based reconstruction and analysis (COBRA). To characterize metabolic phenotypes across bacterial species in the HaPaPro collection, flux balance analysis (FBA) and flux space sampling was performed⁶⁹. Clustering of metabolic phenotypes in probiotics was observed by family (p-value<0.001) using PCA (FIG. 2G). This functional landscape identified opportunities for rational consortium design; for instance, combining Lactobacillacea and Bifidobacteriacea species maximizes metabolic coverage. On the other hand, the close proximity of Akkermansiacea and Bifidobacteriaceae species suggests metabolic redundancy; combining them may not offer any added metabolic functional diversity compared to administering Bifidobacteriacea strains alone. Such mapping allows for the intentional selection of strains that either fill distinct metabolic niches or provide ecological benefits through niche partitioning.

Gaining a deeper understanding of how pathogenic, probiotic, and other host-associated bacteria metabolic phenotypes are similar and different is essential for intentional probiotic strain design. Currently, many pathogenic and host-associated metabolic phenotypes not captured by probiotic species (FIG. 2H), highlighting a significant functional void in the supplement landscape. Importantly, filling this void does not mean a probiotic should broadly mimic the metabolic range of all host-associated species, as many native microbes are associated with dysbiosis. Rather this metabolic gap presents an opportunity for targeted niche occupation. By intentionally expanding a probiotic's metabolic repertoire to specifically overlap with the limiting resources required by a target pathogen, direct resource competition can be engineered. This analysis was expanded upon to demonstrate this concept of targeted competitive exclusion in a specific use case: women's health and the vaginal microbiome.

### Metabolic Landscape of Vaginal Microbes

23 vaginal health supplements utilizing 22 unique probiotic bacterial species were identified; 68% of which were Lactobacillus sp., a dominant member of the healthy vaginal microbiome¹⁵. However, certain populations do not exhibit Lactobacillus dominant community state types (CST) nor symptoms consistent with vaginal dysbiosis²², suggesting homeostatic functions are not restricted to Lactobacillus. To systematically characterize these protective metabolic functions and identify the specific mechanisms preventing pathogen expansion, a combination of computational and in vitro analyses was performed.

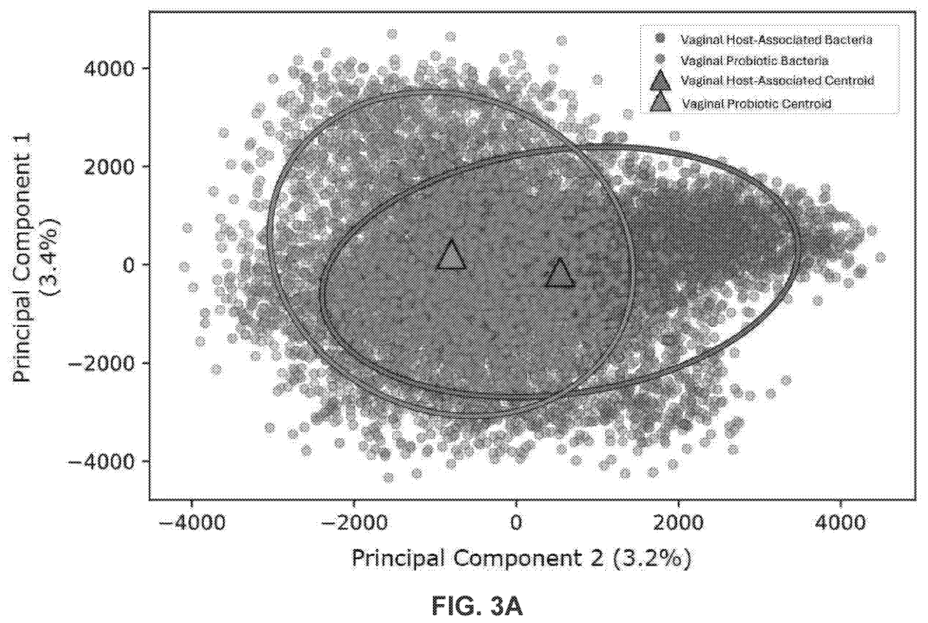

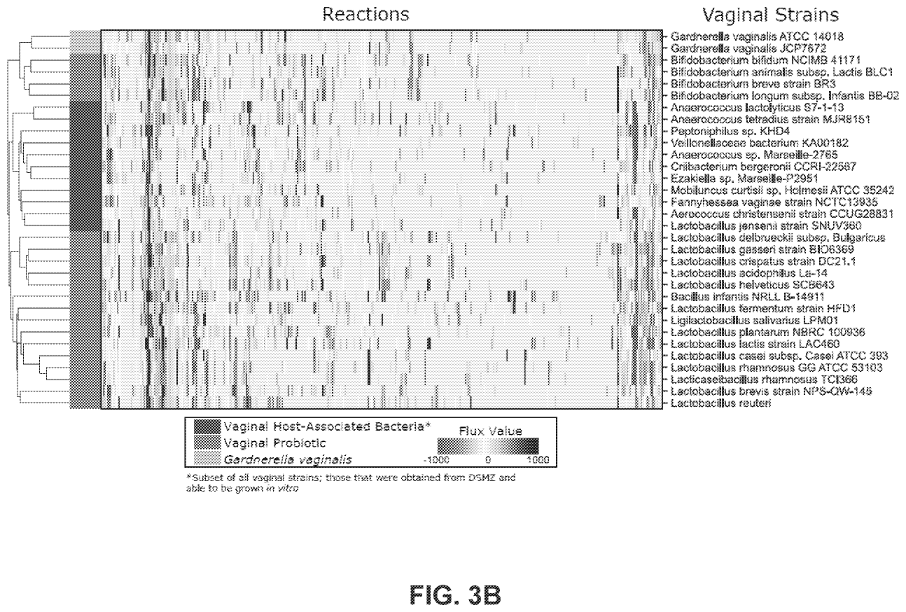

Through dimensionality reduction and visualization of the metabolic phenotypes, it was observed that there are significant functional differences in metabolism between species used as vaginal probiotics in commercially available supplements, and the native flora of the vaginal microbiome, with non-probiotic species covering a wider range of metabolic functions (non-probiotic standard deviation: 897.7; vaginal probiotic standard deviation: 863.35; Kruskal-Wallis test for median distances between centroids: p=2.2e-7) (FIG. 3A). Consistent with a global analysis (FIG. 2H), endogenous species occupy a significantly broader metabolic space, possessing functional traits not captured by commercial probiotic species. This result presents an opportunity to leverage the expanded metabolic repertoire of native vaginal microbes to identify protective functional traits missing in current formulations.

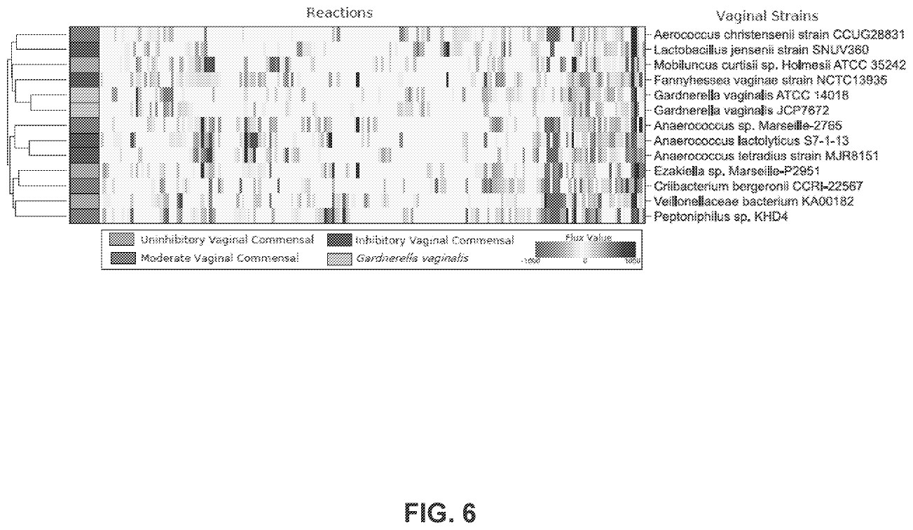

Prevention of Gardnerella spp. growth, frequently associated with BV, is one specific target to consider when designing next-generation probiotics for the vaginal microbiome⁷⁰. To identify potential competitors of Gardnerella spp., the median metabolic signature of two G. vaginalis strains were identified against native vaginal isolates and probiotic species. Importantly, “vaginal probiotics” are species that were included in commercially available probiotic supplements that claim vaginal health benefits. Hierarchical clustering was used to map functional proximity across groups focusing on species with poorly characterized roles in the vaginal niche. For example, Bifidobacteria are widely used as probiotics, but their efficacy as vaginal probiotics has not been well studied (CST IV-C3, Bifidobacterium dominated, uncertain clinical risk). This analysis allowed for the observation of the similarities and differences in metabolic signatures across strains (FIG. 3B, FIG. 6).

G. vaginalis strains exhibited high functional similarity to Bifidobacteria species currently used in vaginal probiotics, yet diverged significantly from the Lactobacillus and Bacillus vaginal probiotic species. This lack of metabolic overlap between the pathobiont and the traditional protective species implies that overlap in function may not be the only driver of resource competition or Lactobacillus-mediated competitive exclusion. Lactobacilli are essential for maintaining vaginal health through secretion of lactic acid to maintain a low pH environment. Results herein suggest that when considering the rational design of probiotics, preventing G. vaginalis colonization may but may not be a more nuanced problem which considers environmental factors in addition to competitive exclusion. To resolve this discrepancy and identify the role of environmental factors, in vitro spent media assays were performed to model competitive interactions between between G. vaginalis and other vaginal microbiome isolates.

Inhibition of G. vaginalis by Vaginal Isolates

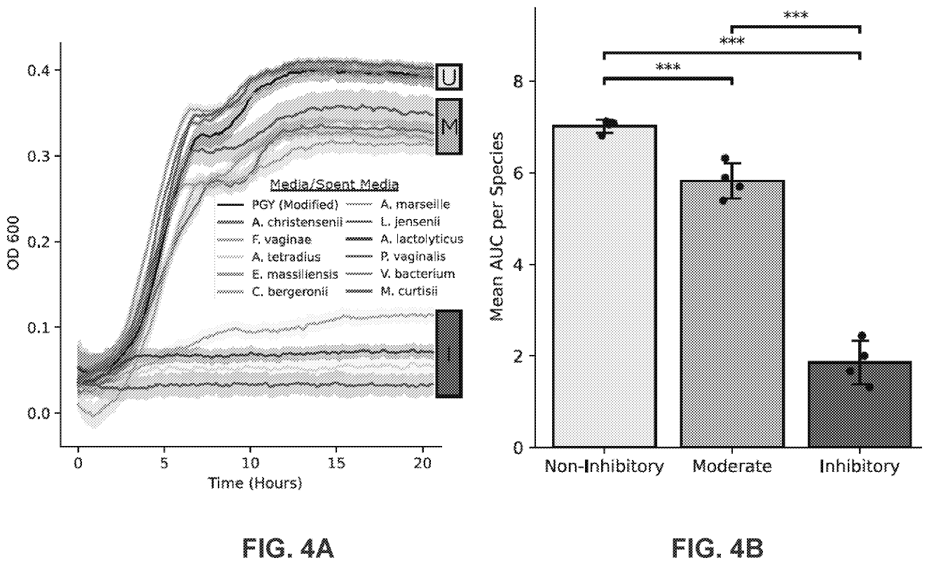

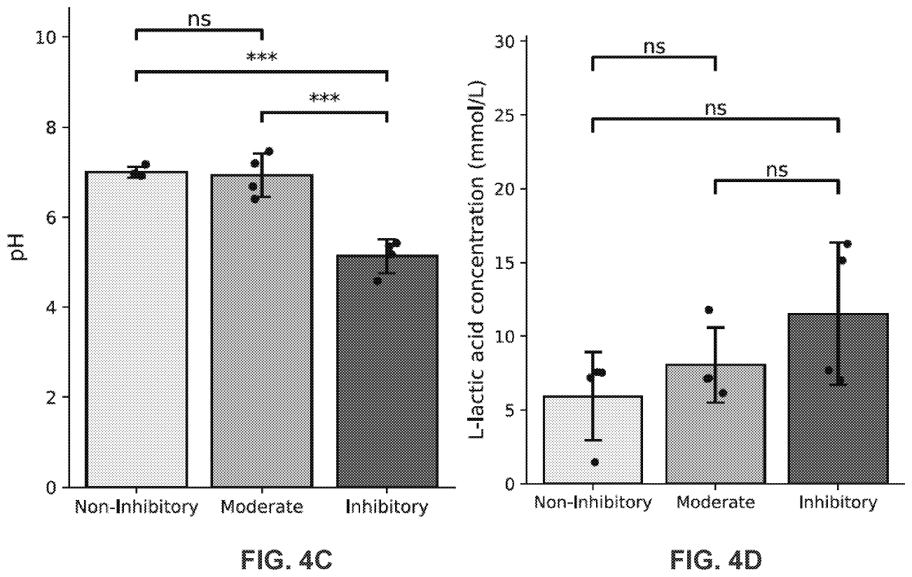

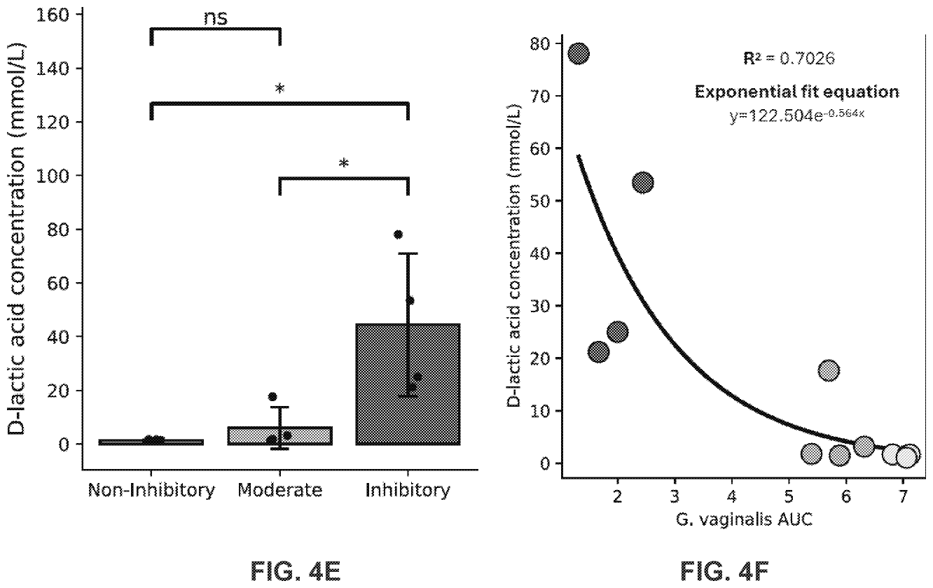

Vaginal microbiome isolates that compete with G. vaginalis could be key candidates for a probiotic consortium to prevent G. vaginalis colonization, if they themselves are not associated with high clinical risk community state types. To further understand differences in metabolic functionality across vaginal microbes and identify which isolates effectively inhibit G. vaginalis growth, an in vitro spent media assay (Methods) was designed and performed as a proxy for assessing competition between vaginal microbes and G. vaginalis. This assay allows one to determine if a stable population of each vaginal isolate produces metabolic byproducts that would inhibit the growth of G. vaginalis. 11 spent media conditions were generated, one for each of the culturable vaginal isolates shown in FIG. 3B, that were able to be cultured in vitro. Then, an overnight culture of G. vaginalis was inoculated into the cell-free supernatants of each isolate to assess the inhibitory potential of secreted metabolic byproducts (FIG. 4A; Methods).

Some variation in the G. vaginalis growth profiles on the 12 media conditions were observed (FIG. 4A). More specifically, three distinct response groups were observed: Non-inhibitory (N), Moderate (M), and Inhibitory (I) (p<0.01 for all pairwise comparisons; FIG. 4B). G. vaginalis grown on non-inhibitory media (Mobiluncus curtisii, Veillonellaceae bacterium, and Ezakiella massiliensis spent media) had the same growth dynamics as G. vaginalis grown on the PGY (Modified) media control. In the moderate group, a significant change in the area under the growth curve of G. vaginalis grown in Peptoniphilus vaginalis, Anaerococcus marseille, Criibacterium bergeronii, and Aerococcus christensenii spent media was observed compared to the uninhibited group (p-value<0.01). In the inhibited group, a statistically significant difference in the area under the growth curve of G. vaginalis grown in Fannyhessea vaginae spent media, Lactobacillus jensenii, Anaerococcus lactolyticus, and Anaerococcus tetradius when compared to the uninhibited (p-value<0.001) and moderate (p-value<0.001) conditions is seen. This potent inhibition suggests diverse mechanisms of suppression, prompting an investigation into whether resource competition or environmental modifiers, such as media acidification, drove the observed exclusion.

D-Lactic Acid Drives G. vaginalis Inhibition

It is well-known that the Lactobacillus dominance in the vaginal microbiome is responsible for protecting against pathogen invasion by maintaining an acidic environment through lactic acid production¹⁵. Significant differences in pH across the spent media of the three growth dynamics groups (N, M, and I) (FIG. 4C) were observed. The spent media that inhibited G. vaginalis growth had a significantly lower pH than the spent media of the moderate and uninhibited groups (p<0.01). However, there was no significant difference between the pH in the uninhibited and moderate groups (p-value>0.8). This result suggests that pH of the spent media plays a major role in inhibiting G. vaginalis growth. Consequently, D- and L-lactic acid concentration was quantified to determine the metabolic basis of this acidification (Methods).

The mmol/L concentrations of L-lactic acid and D-lactic acid determined by the assays are reported in Table 2 below. No significant difference in the concentration of L-lactic acid between the inhibitory, non-inhibitory, and moderate spent media was observed (FIG. 4D). Significant differences in D-lactic acid concentration in the inhibitory spent media condition compared to the moderate and non-inhibitory spent media conditions was observed (FIG. 4E). Additionally, a strong inverse correlation between D-lactic acid concentration and the G. vaginalis area under the curve (AUC (FIG. 4F)) was observed. These results suggest that D-lactic acid production is a key metabolic driver for G. vaginalis growth inhibition in the spent media of L. jensenii, F. vaginae, A. tetradius, and A. lactolyticus.

TABLE 2
Concentration of L-lactic acid, D-lactic acid,
Area Under the Curve G. vaginalis growth

| | G. vaginalis | | L-Lactic | D-Lactic |
|---|---|---|---|---|
| | growth | | Acid | Acid |
| Media | dynamic | G. vaginalis | Conc. | Conc. |
| Condition | category | AUC | (mmol/L) | (mmol/L) |
| PGY (Modified) | Uninhibitory | 6.812 | 7.176 | 1.678 |
| M. curtisii | Uninhibitory | 7.074 | 7.528 | 1.483 |
| E. massiliensis | Uninhibitory | 7.116 | 7.566 | 1.64 |
| V. bacterium | Uninhibitory | 7.055 | 1.453 | 1.052 |
| A. christensenii | Moderate | 5.696 | 11.79 | 17.643 |
| C. bergeronii | Moderate | 5.393 | 7.159 | 1.793 |
| A. marseille | Moderate | 5.886 | 7.128 | 1.476 |
| P. vaginalis | Moderate | 6.315 | 6.144 | 3.156 |
| F. vaginae | Inhibitory | 2.442 | 7.025 | 53.472 |
| A. tetradius | Inhibitory | 1.667 | 15.138 | 21.192 |
| L. jensenii | Inhibitory | 1.315 | 7.689 | 78.059* |
| A. lactolyticus | Inhibitory | 1.999 | 16.263 | 25.016 |

These results are consistent with the general knowledge that Lactobacillus species are typically present in a healthy vagina due to their ability to produce lactic acid and lower vaginal pH. The high D-lactic acid production by F. vaginae, A. tetradius, and A. lactolyticus, demonstrates that pathogen-inhibitory acidification is not exclusive to the Lactobacillus genus. These findings suggest that non-canonical D-lactic acid producers may offer functional equivalence in maintaining a protective vaginal environment. To determine if this inhibitory potential translates to direct competitive exclusion in a shared environment, co-culture assays were performed.

### Characterizing Competitive Interactions

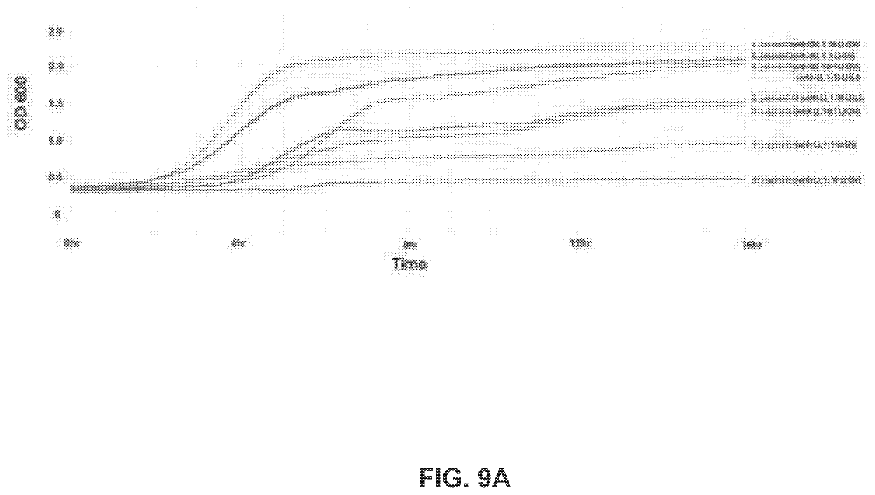

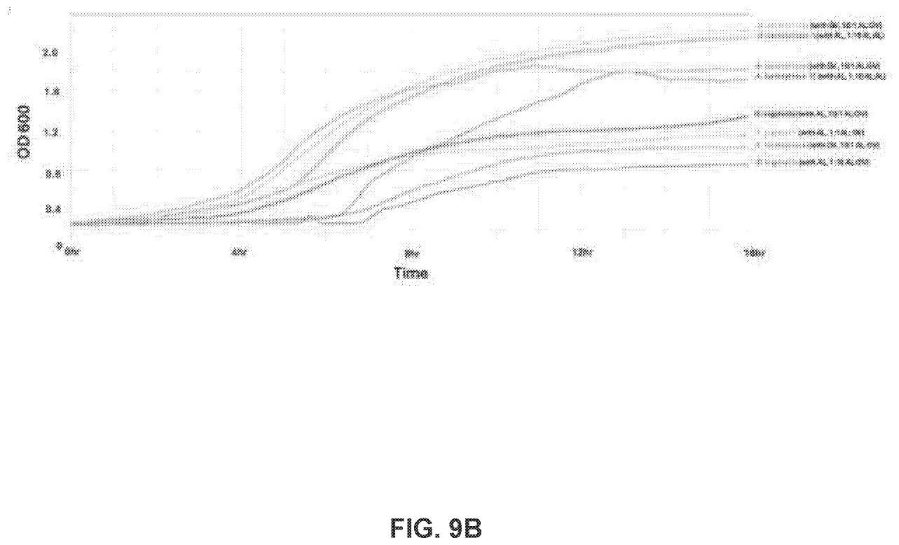

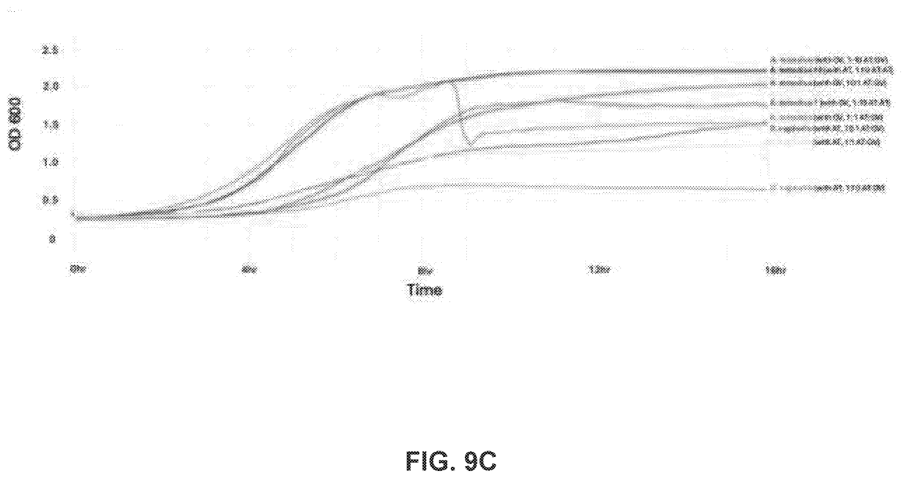

Co-culture assays were performed with G. vaginalis and three high D-lactate producing microbes: L. jensenii, A. lactolyticus, and A. tetradius. L. jensenii. The results of FIGS. 9A-9C show the three high D-lactate producing microbes effectively suppressed the growth of G. vaginalis in co-culture compared to monoculture controls, confirming that its metabolic advantage drives competitive exclusion. Similarly, both A. lactolyticus and A. tetradius exhibited strong suppressive effect, particularly at equal or higher starting abundances. These results demonstrate that D-lactate production, regardless of whether it originates from a health-associated Lactobacillus or a dysbiosis-associated Anaerococcus, is a potent driver of G. vaginalis inhibition in this system. Additionally, the biofilm-forming capacity of the vaginal isolates was investigated. Furthermore, while G. vaginalis exhibited robust biofilm formation relative to F. vaginae, A. tetradius, and A. lactolyticus, the sustained inhibition observed in portioned co-culture confirms this suppression is mediated by secreted soluble factors rather than physical displacement.

To mechanistically elucidate these in vitro interactions, microbial community-scale modeling simulations (MICOM)⁷¹ were utilized to simulate pairwise in silico co-cultures of G. vaginalis with high D-lactate producers: L. jensenii, A. lactolyticus, A. tetradius, and F. vaginae. The models predicted significant competitive pressure on G. vaginalis driven purely by resource competition. When modeled at a 1:1 ratio, L. jensenii reduced the predicted growth rate of G. vaginalis by ~87% relative to the monoculture, a magnitude of suppression that is comparable to that of the BV-associated Anaerococcus species (~89%). Increasing the partner's relative abundance (1:10 ratio of G. vaginalis to partner) yielded a near-total suppression (>98%) of pathogen growth across all pairs, aligning with in vitro co-culture dynamics (FIGS. 9A-9C). Notably, while the model predicted robust suppression when the pathogen was dominant (10:1), experimental data revealed incomplete inhibition at this ratio. This discrepancy demonstrates that while resource competition is a fundamental baseline driver of suppression, successful in vivo exclusion likely depends on reaching a critical threshold of accumulated inhibitory metabolites, such as D-lactate.

### C. Discussion

Despite the proliferation of over-the-counter probiotic products²⁻⁵, a functional rationale for species selection is often absent. The survey of 352 products herein identified only 36 unique species, most of which are marketed without a clear mechanistic link to their intended health benefit. To address this knowledge gap, the HaPaPro collection was developed, utilizing 1,012 genome-scale metabolic models to evaluate the functional diversity of current probiotics relative to native microbes. Analysis revealed that current probiotic species capture only a small subset of the metabolic functions found across pathogenic and host-associated bacteria. This functional gap suggests a significant opportunity to move beyond traditional strain selection toward a design strategy that expands the metabolic niches occupied by probiotic communities.

In the vaginal case example, specific metabolic functions required to inhibit the pathobiont Gardnerella vaginalis were identified. While the protective role of lactic acid is well documented⁷²⁻⁷⁵, this work distinguishes between the isomers, clarifying that D-lactic acid is a key driver of G. vaginalis growth inhibition. Through spent media assays, it was demonstrated that not only Lactobacillus species but also certain non-lactic acid bacteria, including Anaerococcus lactolyticus and A. tetradius, can produce sufficient D-lactate to suppress pathogen growth. This finding shifts the focus from simple acidification to the specific accumulation of therapeutic metabolic byproducts.

Integrating metabolic modeling with in vitro co-cultures (FIGS. 9A-9C) revealed that pathogen suppression is driven by dual mechanisms: resource competition and metabolic inhibition, consistent with previous work⁷⁶. While experimental assays and resulting data discussed herein highlighted D-lactic acid, MICOM simulations demonstrated that nutrient sequestration is a concurrent force. Even in the absence of D-lactate production, the models predicted that L. jensenii could suppress G. vaginalis growth by over 85% simply by outcompeting the pathogen for limiting substrates. This result suggests that the most effective probiotic candidates are those that provide protection through both acidification and efficient niche occupation, preventing pathogen establishment even when metabolic byproduct concentrations are transiently low.

Furthermore, the present example highlights a distinction between metabolic capability and colonization potential⁷⁷. While strong inhibitors were identified, many of these candidates lack the robust biofilm-forming capacity of G. vaginalis. Because bacterial vaginosis is characterized by resilient polymicrobial biofilms^{78,79}, the therapeutic potential of these candidates likely relies on the secretion of diffusible metabolites, like D-lactic acid, that can penetrate the biofilm matrix without requiring the probiotic to physically integrate into the established structure. This distinction is critical for designing probiotics that can effectively displace pathobionts from protected niches.

Clinical context remains essential for translating these mechanistic findings. Although Anaerococcus pp. and F. vaginae exhibited potent inhibition in vitro, they are clinically associated with Community State Type IV (CST IV) and bacterial vaginosis⁸⁰. Their inhibitory capacity likely reflects competitive dynamics with a dysbiotic niche rather than a health-promoting mechanism^{81,82}. Conversely, L. jensenii is a hallmark of the healthy CST V. The convergence of the mechanistic data herein with clinical health associations identifies L. jensenii as the primary actionable candidate for vaginal health interventions.

Looking forward, this systems biology framework provides a scalable foundation for predictive, rationally designed biotherapeutics. While D-lactate drove inhibition in this baseline model, adapting the in silico pipeline to simulate distinct physiological constraints, such as those dictated by specific demographic or clinical parameters, will allow us to uncover alternative, context-dependent metabolic targets. For instance, an analysis of health records revealed that 22% of vaginitis cases (ICD-10-CM: N76.0, N76.1) occur in post-menopausal women, a population with distinct metabolic constraints driven by lower glycogen and estrogen compared to pre-menopausal women (Methods)⁸⁰. Ultimately, the approach presented here informs the rational selection and metabolic engineering of targeted consortia, moving the field beyond empirical strain supplementation toward functional, evidence-based ecological interventions for diverse patient populations.

### Example 2: Further Discussion of Example 1

There has been extensive research on the positive health outcomes linked to probiotic intake, resulting in hundreds of probiotic supplement products being available to consumers over the counter. However, a mechanistic understanding of the bacterial species contained within these supplements and how they can support health of the system of interest is lacking. Consequently, the range of metabolic functions was explored across species in over-the-counter probiotic supplements using a metabolic network modeling approach to identify potential opportunities to expand species used in current supplements to capture a broader range of metabolic functions.

It was observed that the metabolic functions captured across probiotic species used in supplements was only a small subset of the functions captured across all pathogenic and other host-associated species. Furthermore, it is suggested that metabolic network modeling could play a part in rationally designing probiotic combinations, though the specific design goal may vary by clinical context. For general ecosystem restoration, the goal may be to maximize the range of metabolic functions captured to fill available niches and promote stability.

The vaginal microbiome was a focus to identify gaps in probiotics used to support vaginal health. It was observed that most vaginal microbiome-related probiotics consist of lactic acid bacteria like Lactobacilli which are known to support vaginal health by maintaining an acidic vaginal pH. In this work it was desired to identify microbes in the native vaginal flora that prevent G. vaginalis growth, which could be candidates for vaginal probiotic species, assuming the microbes are associated with low clinical risk community state types. Notably, Gardnerella clade is genomically and phenotypically diverse. Through spent media experiments to simulate resource competition between G. vaginalis and other vaginal species, four species (A. lactolyticus, A. tetradius, L. jensenii, and F. vaginae) were identified that inhibit G. vaginalis growth through D-lactic acid production.

Importantly, clinical cohort studies provide the necessary translational context for these findings. While Anaerococcus spp. and F. vaginae showed strong inhibition in assays of the present disclosure, in vivo data consistently places them within Community State Type IV (CST IV), which is strongly associated with bacterial vaginosis and adverse health outcomes. Their ability to inhibit G. vaginalis in vitro likely reflects competitive dynamics within the dysbiotic niche rather than a health-promoting mechanism. Consequently, despite their metabolic potential, they are unsuitable as probiotics. In contrast, L. jensenii is clinically defined as a marker of healthy CST V. The convergence of the mechanistic finding (D-lactate inhibition) with in vivo clinical data (association with health) supports L. jensenii as the primary actionable candidate for this screen.

In this study, only 11 culturable vaginal microbes were used to assess Gardnerella inhibition potential. Selection of these 11 isolates was limited by culturability in vitro, vendor availability, and representation of key vaginal microbiome genera. Because of these limitations, the panel of vaginal microbes used in the present in vitro work is not exhaustive.

The ability of lactic acid to suppress growth of Gardnerella has been previously established and the link between strong Gardnerella competitors and D-lactate has been implied. In this study, it was clarified that D-lactic acid is the isomer most responsible for this inhibition. Additionally, it was demonstrated that non-lactic acid bacteria such as A. lactolyticus, A. tetradius, and F. vaginae can produce large enough quantities of lactic acid to effectively inhibit Gardnerella growth. Currently, lactic acid suppositories are used as a BV treatment. However, the primary limitation of a lactic acid suppository is that it only provides a single transient dose of acid. A live consortium could constantly regenerate lactic acid in the vaginal environment. This finding presents a clear actionable opportunity for metabolic engineering.

To validate whether the metabolic inhibition observed in spent media translates to direct competition, a co-culture assay to examine growth inhibition in real time was utilized. It was observed that high D-lactate producer including L. jensenii, A. lactolyticus, and A. tetradius effectively suppressed G. vaginalis growth in co-culture. This confirms that the inhibitory metabolic byproducts accumulate sufficiently to drive competitive exclusion even in shared environments. Modified PGY medium was utilized for these screens. While this medium does not perfectly mimic the nutritional scarcity of the vaginal environment, it was selected as a permissive condition to support the growth of diverse, fastidious anaerobes that frequently fail to thrive in defined vaginal fluid stimulants. This allowed for a standardized comparison of metabolic potential across species, though future work should validate these interactions in physiologically relevant conditions.

The integration of the present in silico modeling with in vitro co-cultures reveals a multi-layered mechanism of pathogen inhibition. While experimental assays identified D-lactic acid as a primary driver of inhibition, MICOM simulations demonstrate that resource competition is also a likely significant concurrent force. Even without accounting for D-lactate production, models predicted that L. jensenii could suppress G. vaginalis growth by over 85% simply by sequestering shared nutrients. This result suggests that L. jensenii is an efficient competitor for the limiting substrates in the vaginal niche. However, experimental data revealed nuances not captured by the steady-state model; specifically, when G. vaginalis outnumbered Anaerococcus sp. (10:1), it was able to overcome this competitive pressure in culture, whereas the model predicted continued suppression. This discrepancy highlights that while nutrient competition is a potent baseline force, other factors are at play to determine pathogen establishment.

Additionally, In vivo, F. vaginae and G. vaginalis are often found in concert which is a stark contrast to what is seen in these results. The spent media and co-culture assays as well as the community modeling used in this study isolates the effects of secreted metabolic byproducts (like D-lactic acid) and does not capture complex in vivo interactions such as biofilm formation which has been shown to drive synergy between F. vaginae and G. vaginalis. This observation highlights a key limitation of current in vitro models to capture complex community dynamics, which reinforces the need for more complex co-culture experiments in vitro and community-scale models in silico. Further development of physiologically relevant in vitro experimental community dynamic assays is essential for better unraveling of mechanisms of cooperation and inhibition.

The present example highlights a distinction between metabolic capability and colonization potential. While strong metabolic inhibitors of G. vaginalis were identified, experimental characterization revealed that these probiotic candidates generally lack the ability to form robust biofilms compared to G. vaginalis. This presents a significant challenge for therapeutic translation, as bacterial vaginosis is characterized not merely by the presence of G. vaginalis but by complex, polymicrobial biofilms. Recent work by demonstrates that G. vaginalis engages in synergistic interactions with other BV-associated bacteria, such as F. vaginae and P. bivia, to form cooperative, multi-species biofilms that are highly resistant to perturbation. Given that the vaginal microbes tested here are poor biofilm formers, it is possible that they cannot physically displace this established polymicrobial community through competitive exclusion or niche occupation alone. Instead, their therapeutic potential likely relies on the secretion of diffusible metabolites, such as the D-lactic acid identified in work herein, which can penetrate the biofilm matrix and inhibit the growth without requiring the bacteria to physically integrate into the biofilm structure. Additionally, a key strategy for probiotics may not be directly disrupting pathogenic biofilms, rather promoting the persistence of beneficial microbes like Lactobacillus species.

Overall, it is believed that this metabolic network modeling approach combined with in vitro experimentation is a promising step toward rationally designed probiotics for targeted uses, specifically for supporting vaginal microbiome health through G. vaginalis growth inhibition.

Finally, the present results underscore that there should not be a one-size fits-all approach to designing vaginal therapeutics; optimal intervention likely requires a patient-specific framework that accounts for variations in the native vaginal microbiome composition. For instance, post-menopausal women exhibit significantly different vaginal microbiome compositions in both healthy and dysbiotic states driven by changes in systemic estrogen and glycogen availability. Most current BV therapeutics, including the D-lactate mechanism highlighted in this study, are optimized for the glycogen-rich environment of reproductive-age women in min.

### Example 3: Materials and Methods for Example 1

### Survey of Commercial Probiotics

A survey of probiotics available for purchase on the CVS, Walgreens, and Walmart websites, the top three pharmacies in the USA by number of locations (9,554, 9,398, and 6,860 stores respectively)⁵⁷ was perfumed. In this survey, the name of each probiotic supplement product, specified uses (e.g., gut, vaginal, urinary), and all probiotic strains included in each supplement were collected. Additionally, it was determined the total number of strains and the total number of species present in each probiotic supplement. A binary species presence dataframe was created (row: probiotic supplement product, column: probiotic species, 1: species present, 0: species absent). This dataframe was utilized for dimensionality reduction and visualization with PCA (sklearn (version 1.7), matplotlib (version 3.10.7), numpy (version 2.3.5), pandas (version 2.3.3), to observe clustering patterns⁸³.

### Metabolic Network Reconstruction of Pathogenic, Probiotic, and Other Host-Associated Species

1,007 genome scale metabolic network reconstructions (GENREs) of pathogenic, probiotic, and other host-associated bacterial species were generated. To do this, first, genome sequences from the BV-BRC (version 3.52.11)⁸⁴ were selected. One genome sequence for each of the 35 bacterial probiotic species identified in the survey of commercially available probiotics from the BV-BRC was selected. The criteria for selecting probiotic genome sequences were as follows: 1) Select the representative or reference sequence of a given species if it exists, 2) If there is no representative or reference sequence, randomly select a sequence that is considered good quality and complete. Of the 35 probiotic sequences selected, 26 were considered reference or representative sequences, and 9 sequences were considered good and complete without the reference or representative designation. Pathogen sequences were selected in a similar manner. Using the previously published database of metabolic network models as a guide, all reference and representative sequences of the species included in the PATHGENN database⁶⁸ were selected, resulting in 197 pathogenic sequences selected. Finally, other host-associated bacteria to be any species in the BV-BRC database that was not considered pathogenic or probiotic were considered. All reference and representative host-associated sequences that met these criteria were selected, resulting in 775 host-associated sequences.

After selecting the sequences, an annotated protein sequence was generated through an automated pipeline. This annotated protein sequence was used as an input to Reconstructor (version 1.1.2), a tool used for automated GENRE creation⁵⁹. Reconstructor was used to generate genome scale metabolic network reconstructions of all 1,007 sequences. All GENREs were created in the context of the same rich media.

### Reaction and Subsystem Analysis

Reactions were identified that were unique to pathogen, probiotic, and other host-associated bacterial species through model analysis and visualized this data using an upset plot (FIGS. 2B-2C). The list of unique metabolic reactions from each group (pathogenic, probiotic, other host-associated bacteria) was taken, and identified the metabolic subsystem to which they belong. This was done by querying the KEGG (version 115.0) API to identify the general subsystem to which each of these unique reactions belongs⁸⁵. The number of unique reactions in each group that were a part of each metabolic subsystem was then displayed.

### Flux Sampling and Metabolic Phenotype Analysis

All reconstructions were simulated on a rich in silico media containing the following compounds with corresponding modelseed compound IDs: cpd00001 (Water), cpd00035 (L-Alanine), cpd00041 (L-Aspartate), cpd00023 (L-Glutamate), cpd00119 (L-Histidine), cpd00107 (L-Leucine), cpd00060 (L-Methionine), cpd00161 (L-Threonine), cpd00069 (L-Tyrosine), cpd00084 (L-Cysteine), cpd00033 (Glycine), cpd00322 (L-Isoleucine acid), cpd00066 (L-Phenylalanine), cpd00054 (L-Serine), cpd00065 (L-Tryptophan), cpd00156 (L-Valine), cpd00220 (Riboflavin), cpd00644 (Pantothenate), cpd00393 (Folate), cpd00133 (Nicotinamide), cpd00263 (Pyridoxal), cpd00104 (Biotin), cpd00149 (Cobalt), cpd00971 (Sodium), cpd00099 (Chloride), cpd00205 (Potassium), cpd00009 (Phosphate), cpd00063 (Calcium), cpd00254 (Magnesium), cpd10515 (Iron), cpd00030 (Manganese), cpd00242 (Bicarbonate), cpd00226 (Hypoxanthine), cpd01242 (Thyminose), cpd00307 (Cytosine), cpd00092 (Uracil), cpd00117 (D-Alanine), cpd00067 (Hydrogen), cpd00567 (D-Proline), cpd00132 (L-Asparagine), cpd00210 (Taurine), cpd00320 (D-Aspartate), cpd03279 (Deoxyinosine), cpd00246 (Inosine), cpd00311 (Guanosine), cpd00367 (Cytidine), cpd00277 (Deoxyguanosine), cpd00182 (Adenosine), cpd00654 (Deoxycytidine), cpd00412 (Deoxyuridine), cpd00438 (Deoxyadenosine), cpd00274 (Citrulline), cpd00186 (D-Glutamate), cpd00637 (D-Methionine), cpd00105 (D-Ribose), cpd00305 (Thiamin), cpd00309 (Xanthine), cpd00098 (Choline), cpd00207 (Guanine), cpd00082 (D-Fructose), cpd00129 (L-Proline).

The Gapsplit algorithm was used for sampling flux distributions⁸⁶. 500 flux distributions for each GENRE were generated to most accurately capture the range of all metabolic functions (metabolic phenotypes). When comparing flux sampling outputs between taxa the union of all reactions across reconstructions was used, missing reaction fluxes were imputed with zeros for comparison. To visualize similarities and differences in flux distributions across non-pathogen, pathogen, and probiotic species, principal component analysis (PCA) through the sklearn (version 1.7) python (version 3.9.12) package⁸³ was used. Each flux distribution was reduced to a two-dimensional space (two principal components) to plot. In FIG. 2H, pathogenic (middle), probiotics (top) were plotted, and then other host-associated bacterial species (bottom) to best visualize the metabolic phenotype coverage of group. The same method of flux sampling and dimensionality reduction in FIG. 2G and FIG. 3B was used

Heatmap and Hierarchical Clustering of Metabolic Flux Through G. vaginalis, Other Vaginal Microbiome Species, and Vaginal Probiotic Bacterial Species

500 flux distributions per model of interest were generated using Gapsplit (version 1.1.1)⁸⁶. Then, a median flux vector was generated across all 500 samples per each model. All median flux vectors were combined across models of interest into one data frame, removed low variance reactions (variance threshold of 0.1), and dropped highly correlated reactions (correlation threshold of 0.9). After these pre-processing steps, a cluster map was generated using seaborn (version 0.13.2)⁸⁷, clustering on both rows (species-specific models) and columns (reactions) using the Canberra distance. This method was applied to the heatmaps in FIG. 3B and FIG. 6.

### In Vitro Spent Media Assays

16 vaginal microbiome isolates from the DSMZ were obtained: Lactobacillus jensenii strain DSM 20557 (DSM 20557), Aerococcus christensenii strain CCUG28831 (DSM 15819), Criibacterium bergeronii CCRI-22567 (Criibacterium bergeronii CCRI-22567), Ezakiella sp. Marseille-P2951 (DSM 103122), Peptoniphilus sp. KHD2 strain kHD2 (DSM 101742), Anaerococcus sp. Marseille-P2765 (DSM 103343), Corynebacterium fournierii strain Marseille-P2948 (DSM 103271), Corynebacterium accolens DSM 44278 (DSM 44278), Mobiluncus curtsii subsp. Holmesii ATCC 35242 (DSM 21655), Veillonellaceae bacterium KA00182 (DSM 111202), Corynebacterium aurimucosum ATCC 700975 (DSM 44827), Anaerococcus tetradius strain DSM 2951 (DSM 2951), Megasphaera sp. UPII 199-6 (DSM 111201), Anaerococcus lactolyticus DSM 7456 (DSM 7456), Dermabacter vaginalis strain AD1-86 (DSM 100050), Atopobium vaginae strain DSM 15829 (DSM 15829). None of these isolates are found on the list of known misidentified cell lines maintained by the International Cell Line Authentication Committee. These isolates were selected from the larger collection of identified vaginal microbes for several reasons: 1) the isolates were easily obtainable from one reputable source, 2) strains were selected that were specifically isolated from the human vagina. There were strains of some isolates that were available but were not isolated from the vagina; these were not selected.

Ultimately, 11 of these species were robustly grown successfully in anaerobic conditions. After successfully growing these bacterial species in their specified media, a media condition was determined that would successfully grow these 11 species as well as G. vaginalis. It was determined the media that was most successful at robust growth was PGY media (DSMZ) modified with HEPES (6.3 mL) and 10% FBS. Unmodified PGY, NYCIII, and NYCIII media modified with Vitamin K (100 uL) and Hemin (5 mL) was also tried (FIGS. 7A-7C).

After determining a universal media condition (PGY modified; PGY-mod that would lead to maximal growth across isolates, spent media needed collection. Each isolate was inoculated into 10 mL of PGY-mod. For each isolate, this process was repeated 5 times. Each 10 mL aliquot of spent media was filtered sterilized with a 20-mm filter and was pooled to generate 50 mL of spent media per isolate. Spent media was not normalized by culture density.

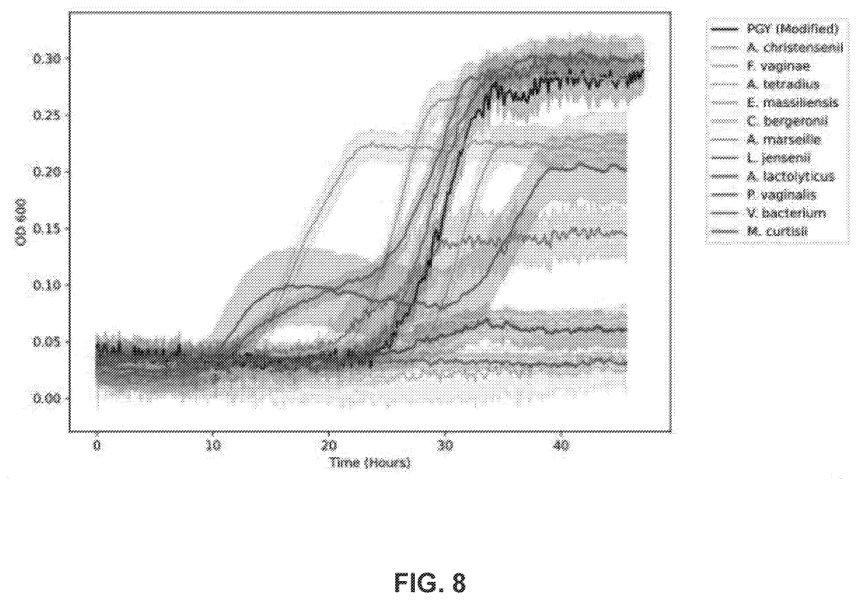

Then, to determine if G. vaginalis would grow on the spent media of each isolate, a spent media assay was performed. This assay involved inoculating G. vaginalis into 5 mL of PGY-mod and was allowed to grow for 24 hours. After 24 hours, the culture was spun down at 6500 rpm for 5 minutes, media was aspirated, and culture was resuspended in fresh PGY-mod media. Then, G. vaginalis was inoculated into the spent media in a 12 well plate at an OD of 0.1 and allowed to grow until stationary phase with continuous growth monitoring using a Cerillo stratus plate reader. This assay was repeated with two different strains of G. vaginalis; GV14018 (results show in FIG. 4A) and JCP7672 (FIG. 8) to ensure the trends observed were consistent across strains. Notably, these two G. vaginalis isolates are from the same clade. Inhibition of G. vaginalis JCP767 in A. tetradius, A. lactolyticus, L. jensenii, and F. vaginae spent media was observed as well. Growth of JCP7672 in the moderate and Non-inhibitory spent media was observed that were mostly consistent with GV14018, but there was some variation (FIG. 8).

### Area Under the Growth Curve Calculations and Identifying Differences in Growth Dynamics

Area under each growth curve in FIG. 4B was calculated using the simps integration method from the scipy package (version 1.16.0)⁸⁸. The mean value was used across replicates for this growth curve calculation. To determine statistical difference between the three observed groups of growth curves, a one-way ANOVA test, with subsequent pair-wise t-tests between groups, with a significance value of 0.05 (n=3 per condition) was used.

### Spent Media pH Measurements and Differences Between Groups

A pH meter (Accumet basic AB15 pH meter) was used to determine the pH of each media condition used in the spent media assay with accuracy to 0.01. Then, to determine statistically significant differences in pH between the three growth dynamics groups pairwise Welch's t-tests was used, with a significance value of 0.05.

### L-Lactic Acid and D-Lactic Acid Assays

Concentrations of D-lactic acid and L-lactic acid were quantified using the Novus Biologicals D-Lactic Acid/Lactate Assay Kit (Colorimetric) NBP3-25788 and L-lactic Acid Assay Kit (Colorimetric) NBP3-25875 respectively (n=2 per spent media condition). After completing the specified kit protocol, OD was read at 530 nm using a TECAN plate reader. The assay was re-run (standards and samples) at dilution for samples that read above the standard curve range. D-lactic acid and L-lactic acid concentrations were computed according to the standard curve.

### Co-Culture Assay

Cultures that produced the most lactic acid were grown under anaerobic conditions at 37 °C in modified PGY (described above), absorbance at 600 nm (A₆₀₀) measured and cultures adjusted to a starting density of 0.05-0.005 in fresh modified PGY. Co-culture conditions included strains at 1:1 or 10:1 ratio where each side of a Duet co-culture plate (Cerillo) contained 450 uL with one strain grown on each side. Plates were sealed using a Breathe-Easy membrane (USA scientific) and growth monitored at A₆₀₀ with shaking (Alto plate reader; Cerillo) under anaerobic conditions (FIGS. 9A-9C).

### Biofilm Assay

Duets used in the co-culture assay described above were used to measure biofilm with the crystal violet assay. Briefly, cultures were gently aspirated off and plates allowed to dry in a biosafety cabinet (5 min). Biofilms were fixed with 75% ethanol (10 min), stained with 0.5% Crystal violet (5 min), rinsed with water, and biofilm-associated crystal violet solubilized with 95% ethanol and A₅₇₀ measured.

### Community Modeling

Pairwise community modeling was performed using the MICOM (Microbial community modeling, micom version 0.31.1)⁷¹ framework to simulate interactions between Gardnerella vaginalis and potential inhibitory partners. For each pairwise comparison, community models were constructed by combining genome-scale metabolic models of G. vaginalis with high-lactate producing vaginal microbes (L. jensenii, A. lactolyticus, A. tetradius, F. vaginae). Community simulations were performed using the cooperative tradeoff optimization algorithm (fraction=0.5), which balances individual organism growth objectives with community-level fitness through quadratic programming optimization (solved using OSQP). Each pairwise interaction was simulated at three different initial relative abundance rations (1:1, 1:10, and 10:1, representing G. vaginalis:partner) to assess how competitive dynamics vary with numerical dominance. From each simulation, the growth suppression was calculated as the fractional reduction in G. vaginalis growth rate relative to its growth in monoculture to identify key metabolic features associated with effective G. vaginalis suppression and to inform engineering targets for probiotic strain development.

### TriNetX Cohort Exploration

To explore the number of vaginitis cases presented in the UVA health system, TriNetX was used. The data used in this study was collected on Jun. 5, 2025 from the TriNetX University of Virginia Network, which provided access to electronic medical records (diagnoses, procedures, medications, laboratory values, genomic information) from 730,801 patients in the health care organization. This retrospective study is exempt from informed consent. The data reviewed is a secondary analysis of existing data, does not involve intervention or interaction with human subjects, and is de-identified per the de-identification standard defined in Section § 164.514(a) of the HIPAA Privacy rule. The process by which the data is de-identified is attested to through a formal determination by a qualified expert as defined in section § 164.514(b)(1) of the HIPAA Privacy Rule. This formal determination by a qualified expert refreshed on December 2020.

### Code Availability

The code generated during the current study are available at the following DOI: doi.org/10.5281/zenodo.19338012, the contents of the website are incorporated by reference as if fully set forth herein.

### REFERENCES RELATED TO THE PRESENT DISCLOSURE

1. Hill, C. et al. Expert consensus document. The International Scientific Association for Probiotics and Prebiotics consensus statement on the scope and appropriate use of the term probiotic. Nat. Rev. Gastroenterol. Hepatol. 11, 506-514 (2014).
2. Song, E. et al. Effects of kimchi on human health: a scoping review of randomized controlled trials. J. Ethn. Foods 10, 7 (2023).
3. González, S. et al. Fermented Dairy Foods: Impact on Intestinal Microbiota and Health-Linked Biomarkers. Front. Microbiol. 10, (2019).
4. Batista, P., Penas, M. R., Pintado, M. & Oliveira-Silva, P. Kombucha: Perceptions and Future Prospects. Foods Basel Switz. 11, 1977 (2022).
5. Raak, C., Ostermann, T., Boehm, K. & Molsberger, F. Regular Consumption of Sauerkraut and Its Effect on Human Health: A Bibliometric Analysis. Glob. Adv. Health Med. 3, 12-18 (2014).
6. Cuamatzin-García, L. et al. Traditional Fermented Foods and Beverages from around the World and Their Health Benefits. Microorganisms 10, 1151 (2022).
7. Blair, H. A. SER-109 (VOWST™): A Review in the Prevention of Recurrent Clostridioides difficile Infection. Drugs 84, 329-336 (2024).
8. Doosetty, S. et al. Efficacy of Fecal Microbiota (REBYOTA) in Recurrent Clostridium difficile Infections: A Systematic Review and Meta-Analysis. Cureus 16, e58862 (2024).
9. Kothari, D., Patel, S. & Kim, S.-K. Probiotic supplements might not be universally-effective and safe: A review. Biomed. Pharmacother. 111, 537-547 (2019).
10. Venugopalan, V., Shriner, K. A. & Wong-Beringer, A. Regulatory Oversight and Safety of Probiotic Use. Emerg. Infect. Dis. 16, 1661-1665 (2010).
11. López-Moreno, A. & Aguilera, M. Vaginal Probiotics for Reproductive Health and Related Dysbiosis: Systematic Review and Meta-Analysis. J. Clin. Med. 10, 1461 (2021).
12. Mashatan, N. et al. Probiotics in vaginal health. Pathog. Dis. 81, ftad012 (2023).
13. Mei, Z. & Li, D. The role of probiotics in vaginal health. Front. Cell. Infect. Microbiol. 12, 963868 (2022).
14. van de Wijgert, J. & Verwijs, M. C. Lactobacilli-containing vaginal probiotics to cure or prevent bacterial or fungal vaginal dysbiosis: a systematic review and recommendations for future trial designs. BJOG Int. J. Obstet. Gynaecol. 127, 287-299 (2020).
15. France, M., Alizadeh, M., Brown, S., Ma, B. & Ravel, J. Towards a deeper understanding of the vaginal microbiota. Nat. Microbiol. 7, 367-378 (2022).
16. Valeriano, V. D. et al. Vaginal dysbiosis and the potential of vaginal microbiome-directed therapeutics. Front. Microbiomes 3, (2024).
17. Gardner, H. L. & Dukes, C. D. Haemophilus vaginalis vaginitis: A newly defined specific infection previously classified “nonspecific” vaginitis. Am. J. Obstet. Gynecol. 69, 962-976 (1955).
18. Mondal, A. S., Sharma, R. & Trivedi, N. Bacterial vaginosis: A state of microbial dysbiosis. Med. Microecol. 16, 100082 (2023).
19. Vodstrcil, L. A., Muzny, C. A., Plummer, E. L., Sobel, J. D. & Bradshaw, C. S. Bacterial vaginosis: drivers of recurrence and challenges and opportunities in partner treatment. BMC Med. 19, 194 (2021).
20. Bradshaw, C. S. et al. High recurrence rates of bacterial vaginosis over the course of 12 months after oral metronidazole therapy and factors associated with recurrence. J. Infect. Dis. 193, 1478-1486 (2006).
21. Muzny, C. A. & Sobel, J. D. The Role of Antimicrobial Resistance in Refractory and Recurrent Bacterial Vaginosis and Current Recommendations for Treatment. Antibiotics 11, 500 (2022).
22. Li, W. & Ma, Z. (Sam). Dominance network analysis of the healthy human vaginal microbiome not dominated by Lactobacillus species. Comput. Struct. Biotechnol. J. 18, 3447-3456 (2020).
23. Vivekanandan, V. et al. VagiBIOM Lactobacillus suppository improves vaginal health index in perimenopausal women with bacterial vaginosis: a randomized control trial. Sci. Rep. 14, 3317 (2024).
24. Qi, F. et al. Orally administrated Lactobacillus gasseri TM13 and Lactobacillus crispatus LG55 can restore the vaginal health of patients recovering from bacterial vaginosis. Front. Immunol. 14, 1125239 (2023).
25. Schwebke, J. R. & Desmond, R. A. Tinidazole vs metronidazole for the treatment of bacterial vaginosis. Am. J. Obstet. Gynecol. 204, 211.e1-6 (2011).
26. Schwebke, J. R. & Desmond, R. A. A randomized trial of the duration of therapy with metronidazole plus or minus azithromycin for treatment of symptomatic bacterial vaginosis. Clin. Infect. Dis. Off. Publ. Infect. Dis. Soc. Am. 44, 213-219 (2007).
27. Vujic, G., Jajac Knez, A., Despot Stefanovic, V. & Kuzmic Vrbanovic, V. Efficacy of orally applied probiotic capsules for bacterial vaginosis and other vaginal infections: a double-blind, randomized, placebo-controlled study. Eur. J. Obstet. Gynecol. Reprod. Biol. 168, 75-79 (2013).
28. Voorspoels, J., Casteels, M., Remon, J. P. & Temmerman, M. Local treatment of bacterial vaginosis with a bioadhesive metronidazole tablet. Eur. J. Obstet. Gynecol. Reprod. Biol. 105, 64-66 (2002).
29. Verwijs, M. C., Agaba, S. K., Darby, A. C. & van de Wijgert, J. H. H. M. Impact of oral metronidazole treatment on the vaginal microbiota and correlates of treatment failure. Am. J. Obstet. Gynecol. 222, 157.e1-157.e13 (2020).
30. Thulkar, J., Kriplani, A. & Agarwal, N. Probiotic and metronidazole treatment for recurrent bacterial vaginosis. Int. J. Gynaecol. Obstet. Off. Organ Int. Fed. Gynaecol. Obstet. 108, 251-252 (2010).
31. Sobel, J. D. et al. Prognostic Indicators of Recurrence of Bacterial Vaginosis. J. Clin. Microbiol. 57, e00227-19 (2019).
32. Shalev, E., Battino, S., Weiner, E., Colodner, R. & Keness, Y. Ingestion of yogurt containing Lactobacillus acidophilus compared with pasteurized yogurt as prophylaxis for recurrent candidal vaginitis and bacterial vaginosis. Arch. Fam. Med. 5, 593-596 (1996).
33. Reid, G., Burton, J., Hammond, J.-A. & Bruce, A. W. Nucleic Acid-Based Diagnosis of Bacterial Vaginosis and Improved Management Using Probiotic Lactobacilli. J. Med. Food 7, 223-228 (2004).
34. Raja, I., Basavareddy, A., Mukherjee, D. & Meher, B. Randomized, double-blind, comparative study of oral metronidazole and tinidazole in treatment of bacterial vaginosis. Indian J. Pharmacol. 48, 654 (2016).
35. Parent, D. et al. Therapy of bacterial vaginosis using exogenously-applied Lactobacilli acidophili and a low dose of estriol: a placebo-controlled multicentric clinical trial. Arzneimittelforschung. 46, 68-73 (1996).
36. Paavonen, J., Mangioni, C., Martin, M. A. & Wajszczuk, C. P. Vaginal clindamycin and oral metronidazole for bacterial vaginosis: a randomized trial. Obstet. Gynecol. 96, 256-260 (2000).
37. Anukam, K. et al. Augmentation of antimicrobial metronidazole therapy of bacterial vaginosis with oral probiotic Lactobacillus rhamnosus GR-1 and Lactobacillus reuteri RC-14: randomized, double-blind, placebo controlled trial. Microbes Infect. 8, 1450-1454 (2006).
38. Neri, A., Sabah, G. & Samra, Z. Bacterial vaginosis in pregnancy treated with yoghurt. Acta Obstet. Gynecol. Scand. 72, 17-19 (1993).
39. Mastromarino, P., Vitali, B. & Mosca, L. Bacterial vaginosis: a review on clinical trials with probiotics. New Microbiol. 36, 229-238 (2013).
40. Martinez, R. C. R. et al. Improved cure of bacterial vaginosis with single dose of tinidazole (2 g), Lactobacillus rhamnosus GR-1, and Lactobacillus reuteri RC-14: a randomized, double-blind, placebo-controlled trial. Can. J. Microbiol. 55, 133-138 (2009).
41. Ling, Z. et al. The Restoration of the Vaginal Microbiota After Treatment for Bacterial Vaginosis with Metronidazole or Probiotics. Microb. Ecol. 65, 773-780 (2013).
42. Larsson, P.-G. et al. Extended antimicrobial treatment of bacterial vaginosis combined with human lactobacilli to find the best treatment and minimize the risk of relapses. BMC Infect. Dis. 11, 223 (2011).
43. Larsson, P.-G., Stray-Pedersen, B., Ryttig, K. R. & Larsen, S. Human lactobacilli as supplementation of clindamycin to patients with bacterial vaginosis reduce the recurrence rate; a 6-month, double-blind, randomized, placebo-controlled study. BMC Womens Health 8, 3 (2008).
44. Kurkinen-Raty, M. et al. A randomised controlled trial of vaginal clindamycin for early pregnancy bacterial vaginosis. BJOG Int. J. Obstet. Gynaecol. 107, 1427-1432 (2000).
45. Kovachev, S. & Vatcheva-Dobrevski, R. [Efficacy of combined 5-nitroimidazole and probiotic therapy of bacterial vaginosis: randomized open trial]. Akush. Ginekol. (Sofia) 52, 19-26 (2013).
46. Kekki, M. et al. Vaginal clindamycin in preventing preterm birth and peripartal infections in asymptomatic women with bacterial vaginosis: a randomized, controlled trial. Obstet. Gynecol. 97, 643-648 (2001).
47. Hantoushzadeh, S. et al. Comparative efficacy of probiotic yoghurt and clindamycin in treatment of bacterial vaginosis in pregnant women: A randomized clinical trial. J. Matern. Fetal Neonatal Med. 25, 1021-1024 (2012).
48. Hallén, A., Jarstrand, C. & Påhlson, C. Treatment of bacterial vaginosis with lactobacilli. Sex. Transm. Dis. 19, 146-148 (1992).
49. Fredricsson, B., Englund, K., Weintraub, L., Olund, A. & Nord, C. E. Bacterial vaginosis is not a simple ecological disorder. Gynecol. Obstet. Invest. 28, 156-160 (1989).
50. Eriksson, K., Carlsson, B., Forsum, U. & Larsson, P. A double-blind treatment study of bacterial vaginosis with normal vaginal lactobacilli after an open treatment with vaginal clindamycin ovules. Acta Derm. Venereol. 85, 42-46 (2005).
51. Darwish, A., Elnshar, E. M., Hamadeh, S. M. & Makarem, M. H. Treatment options for bacterial vaginosis in patients at high risk of preterm labor and premature rupture of membranes. J. Obstet. Gynaecol. Res. 33, 781-787 (2007).
52. Brandt, M. et al. Intravaginally applied metronidazole is as effective as orally applied in the treatment of bacterial vaginosis, but exhibits significantly less side effects. Eur. J. Obstet. Gynecol. Reprod. Biol. 141, 158-162 (2008).
53. Bradshaw, C. S. et al. Efficacy of Oral Metronidazole with Vaginal Clindamycin or Vaginal Probiotic for Bacterial Vaginosis: Randomised Placebo-Controlled Double-Blind Trial. PLoS ONE 7, e34540 (2012).
54. Bohbot, J. M. et al. Efficacy and safety of vaginally administered lyophilized Lactobacillus crispatus IP 174178 in the prevention of bacterial vaginosis recurrence. J. Gynecol. Obstet. Hum. Reprod. 47, 81-86 (2018).
55. Anukam, K. C. et al. Clinical study comparing probiotic Lactobacillus GR-1 and RC-14 with metronidazole vaginal gel to treat symptomatic bacterial vaginosis. Microbes Infect. 8, 2772-2776 (2006).
56. Heirendt, L. et al. Creation and analysis of biochemical constraint-based models using the COBRA Toolbox v.3.0. Nat. Protoc. 14, 639-702 (2019).
57. Pharmacy Near Me®: Local Pharmacies & Doctors—Locator & Guide. Pharmacy Near Me https://pharmacy-near-me.com/(2017).
58. Kwoji, I. D., Aiyegoro, O. A., Okpeku, M. & Adeleke, M. A. Multi-Strain Probiotics: Synergy among Isolates Enhances Biological Activities. Biology 10, 322 (2021).
59. Jenior, M. L., Glass, E. M. & Papin, J. A. Reconstructor: a COBRApy compatible tool for automated genome-scale metabolic network reconstruction with parsimonious flux-based gap-filling. Bioinformatics 39, btad367 (2023).
60. Chen, Y. & Fang, J.-Y. The role of colonic microbiota amino acid metabolism in gut health regulation. Cell Insight 4, 100227 (2025).
61. Leitão-Gonçalves, R. et al. Commensal bacteria and essential amino acids control food choice behavior and reproduction. PLOS Biol. 15, e2000862 (2017).
62. Risdian, C., Mozef, T. & Wink, J. Biosynthesis of Polyketides in Streptomyces. Microorganisms 7, 124 (2019).
63. Onwueme, K. C., Vos, C. J., Zurita, J., Ferreras, J. A. & Quadri, L. E. N. The dimycocerosate ester polyketide virulence factors of mycobacteria. Prog. Lipid Res. 44, 259-302 (2005).
64. Pugh, J. N. et al. Probiotic supplementation increases carbohydrate metabolism in trained male cyclists: a randomized, double-blind, placebo-controlled crossover trial. Am. J. Physiol. Endocrinol. Metab. 318, E504-E513 (2020).
65. Baba, Y., Tsuge, D. & Aoki, R. Enhancement of carbohydrate metabolism by probiotic and prebiotic intake promotes short-chain fatty acid production in the gut microbiome: A randomized, double-blind, placebo-controlled crossover trial. Biosci. Biotechnol. Biochem. zbaf071 (2025) doi:10.1093/bbb/zbaf071.
66. Markowiak-Kopeć, P. & Śliżewska, K. The Effect of Probiotics on the Production of Short-Chain Fatty Acids by Human Intestinal Microbiome. Nutrients 12, 1107 (2020).
67. Vinolo, M. A. R., Rodrigues, H. G., Nachbar, R. T. & Curi, R. Regulation of Inflammation by Short Chain Fatty Acids. Nutrients 3, 858-876 (2011).
68. Glass, E. M., Dillard, L. R., Kolling, G. L., Warren, A. S. & Papin, J. A. Niche-specific metabolic phenotypes can be used to identify antimicrobial targets in pathogens. PLOS Biol. 22, e3002907 (2024).
69. Herrmann, H. A., Dyson, B. C., Vass, L., Johnson, G. N. & Schwartz, J.-M. Flux sampling is a powerful tool to study metabolism under changing environmental conditions. Npj Syst. Biol. Appl. 5, 1-8 (2019).
70. Morrill, S., Gilbert, N. M. & Lewis, A. L. Gardnerella vaginalis as a Cause of Bacterial Vaginosis: Appraisal of the Evidence From in vivo Models. Front. Cell. Infect. Microbiol. 10, 168 (2020).
71. Diener, C., Gibbons, S. M. & Resendis-Antonio, O. MICOM: Metagenome-Scale Modeling To Infer Metabolic Interactions in the Gut Microbiota. mSystems 5, 10.1128/msystems.00606-19 (2020).
72. O'Hanlon, D. E., Moench, T. R. & Cone, R. A. In vaginal fluid, bacteria associated with bacterial vaginosis can be suppressed with lactic acid but not hydrogen peroxide. BMC Infect. Dis. 11, 200 (2011).
73. Manhanzva, M. T. et al. Inflammatory and antimicrobial properties differ between vaginal Lactobacillus isolates from South African women with non-optimal versus optimal microbiota. Sci. Rep. 10, 6196 (2020).
74. Mahmoud, M. Y. et al. Lactobacillus crispatus-loaded electrospun fibers yield viable and metabolically active bacteria that kill Gardnerella in vitro. Eur. J. Pharm. Biopharm. Off. J. Arbeitsgemeinschaft Pharm. Verfahrenstechnik EV 187, 68-75 (2023).
75. Chetwin, E. et al. Antimicrobial and inflammatory properties of South African clinical Lactobacillus isolates and vaginal probiotics. Sci. Rep. 9, 1917 (2019).
76. Dillard, L. R., Glass, E. M., Lewis, A. L., Thomas-White, K. & Papin, J. A. Metabolic Network Models of the Gardnerella Pangenome Identify Key Interactions with the Vaginal Environment. mSystems 8, e0068922 (2023).
77. Walter, J., Maldonado-Gómez, M. X. & Martínez, I. To engraft or not to engraft: An Ecological Framework for Gut Microbiome Modulation with Live Microbes. Curr. Opin. Biotechnol. 49, 129-139 (2018).
78. Rosca, A. S., Castro, J., França, Â., Vaneechoutte, M. & Cerca, N. Gardnerella vaginalis Dominates Multi-Species Biofilms in both Pre-Conditioned and Competitive In Vitro Biofilm Formation Models. Microb. Ecol. 84, 1278-1287 (2022).
79. Sousa, L. G. V., Novak, J., França, A., Muzny, C. A. & Cerca, N. Gardnerella vaginalis, Fannyhessea vaginae, and Prevotella bivia Strongly Influence Each Other's Transcriptome in Triple-Species Biofilms. Microb. Ecol. 87, 117 (2024).
80. Mitchell, C. M. et al. Bacterial Communities Associated With Abnormal Nugent Score in Postmenopausal Versus Premenopausal Women. J. Infect. Dis. 223, 2048-2052 (2021).
81. Human Microbiome Project Consortium. Structure, function and diversity of the healthy human microbiome. Nature 486, 207-214 (2012).
82. Ravel, J. et al. Vaginal microbiome of reproductive-age women. Proc. Natl. Acad. Sci. 108, 4680-4687 (2011).
83. Pedregosa, F. et al. Scikit-learn: Machine Learning in Python. J. Mach. Learn. Res. 12, 2825-2830 (2011).
84. Olson, R. D. et al. Introducing the Bacterial and Viral Bioinformatics Resource Center (BV-BRC): a resource combining PATRIC, IRD and ViPR. Nucleic Acids Res. 51, D678-D689 (2023).
85. Kanehisa, M. & Goto, S. KEGG: kyoto encyclopedia of genes and genomes. Nucleic Acids Res. 28, 27-30 (2000).
86. Keaty, T. C. & Jensen, P. A. Gapsplit: efficient random sampling for non-convex constraint-based models. Bioinformatics 36, 2623-2625 (2020).
87. Waskom, M. L. seaborn: statistical data visualization. J. Open Source Softw. 6, 3021 (2021).
88. Virtanen, P. et al. SciPy 1.0: fundamental algorithms for scientific computing in Python. Nat. Methods 17, 261-272 (2020).

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1) A method of identifying candidate probiotics for administration against a candidate bacterial infection, comprising:
identifying one or more candidate probiotic species;
identifying one or more candidate pathogenic bacterial species;
identifying one or more candidate host bacterial species;
selecting a representative genome of each of the one or more candidate probiotic bacterial species, one or more of candidate host bacterial species, and one or more candidate host bacterial species;
generating an annotated protein sequence for each representative genome by assigning functional pathways to a set of proteins encoded by each representative genome;
determining superorganism-level functional metabolic capacity from each of the annotated protein sequences of each of the one or more candidate probiotic bacterial species, one or more candidate host bacterial species, and one or more candidate host bacterial species;
identifying metabolic reactions with differential metabolic capacities from the determined superorganism-level functional metabolic capacities between the one or more candidate probiotic bacterial species, one or more candidate pathogenic bacterial species, and one or more candidate host bacterial species;
clustering the identified metabolic reactions with differential metabolic capacities according to a metabolic subsystem;
characterizing the metabolic phenotype from the identified metabolic reactions with differential metabolic capacities from each of the representative genomes of each of the one or more candidate probiotic bacterial species, one or more of candidate host bacterial species, and one or more candidate host bacterial species;
clustering the characterized metabolic phenotypes; and
selecting one or more probiotic species from the one or more candidate probiotic bacterial species according to clustered characterized metabolic phenotypes that would metabolically compete with the one or more candidate pathogenic bacterial species.

2) The method of claim 1, wherein the one or more candidate host bacterial species are identified from an anatomical area of a subject to infection by the one more candidate pathogenic bacterial species.

3) The method of claim 1, wherein the one or more candidate pathogenic bacterial species cause a Bacterial Vaginosis (BV) diagnosis in a subject.

4) The method of claim 1, wherein the one or more candidate host bacterial species are identified from a vaginal swab from one or more subjects having or suspected of having Bacterial Vaginosis (BV).

5) The method of claim 1, wherein the one or more candidate pathogenic bacterial species comprise a Gardnerella spp. strain.

6) The method of claim 5, wherein the Gardnerella spp. strain comprises G. vaginalis.

7) The method of claim 1, wherein the selecting is by selecting a representative reference sequence from a database, or, if none exists, randomly selecting a representative sequence that is considered good and complete.

8) The method of claim 1, wherein the superorganism-level functional capacity is determined by:
mapping metagenomic reads of each representative genome to the annotated proteins; and
differentiating functionalities of each mapped metagenomic read.

9) The method of claim 1, wherein determining superorganism-level functional metabolic capacity is with genome-scale metabolic network reconstructions (GENREs).

10) The method of claim 1, wherein clustering the identified metabolic reactions with differential metabolic capacities according to a metabolic subsystem comprises:
annotating the gene content of each cluster;
quantifying the annotated gene content;
segregating the quantified gene content based on one or more candidate probiotic bacterial species, one or more of candidate host bacterial species, and one or more candidate host bacterial species;
identifying genes enriched in one or more metabolic pathways from the segregated quantified gene content of the one or more candidate probiotic bacterial species compared to the one or more of candidate host bacterial species and the one or more candidate host bacterial species;
reconstructing the metabolism of the one or more metabolic pathways from the quantified gene content with gene identities mapped onto the one or more candidate probiotic bacterial species, the one or more of candidate host bacterial species, and the one or more candidate host bacterial species.

11) The method of claim 1, wherein the metabolic subsystem comprises one or more of glycan, secondary metabolites, energy, terpenoids and polyketides, xenobiotics, nucleotide, lipid, cofactors and vitamins, carbohydrate, or amino acid.

12) The method of claim 1, wherein characterizing the metabolic phenotype is with flux balance analysis (FBA) and flux space sampling.

13) The method of claim 1, wherein characterizing the metabolic phenotype comprises segregating the reconstructed metabolism of each representative genome into cooperative or competitive secondary candidate bacterial species in relation to the one or more candidate pathogenic bacterial species.

14) The method of claim 1, further comprising:
culturing the one or more selected probiotic bacterial species in the presence of cell culture media to create spent probiotic media comprising metabolic byproducts derived from the one or more selected probiotic bacterial species;
culturing the one or more identified candidate pathogenic bacterial species separately from the one or more selected probiotic bacterial species;
administering the spent probiotic media to the one or more identified candidate pathogenic bacterial species cultures;
determining a level of growth of the one or more identified candidate pathogenic bacterial species in the presence of the spent probiotic media.

15) The method of claim 13, furthering comprising:
culturing the one or more selected host bacterial species in the presence of cell culture media to create spent host media comprising metabolic byproducts derived from the one or more selected host bacterial species; and
administering the spent host media to the one or more identified candidate pathogenic bacterial species cultures before, during, or after the administration of the spent probiotic media;
determining a level of growth of the one or more identified candidate pathogenic bacterial species in the presence of the spent probiotic media and spent host media.

16) The method of claim 14, wherein the one or more selected probiotic bacterial species and one or more host bacterial species are co-cultured to create a spent mixed media comprising metabolic byproducts of both the one or more selected probiotic bacterial species and one or more host bacterial species.

17) The method of claim 14, further comprising determining the levels of the metabolic byproducts in the spent media.

18) The method of claim 14, further comprising measuring the pH of the spent media.

19) A computer-readable medium, comprising:
machine-readable instructions stored in the memory that, when executed by a processor of a computing device, cause the computing device to at least:
carry out the method of claim 1.

20) A system, comprising:
a computing device comprising a processor and a memory; and
machine-readable instructions stored in the memory that, when executed by the processor, cause the computing device to at least:
carry out the method of claim 1.
